SEQUENCE LISTING (38 sequences)

SEQ ID NO: 1 - protein, 55 aa, Bos taurus
CADVTLEGQVYPGKGGGSKEKNQTKQEKGKKKKERDLKPRASKEDNRAGSKKEEL

SEQ ID NO: 2 - protein, 57 aa, Canis familiaris
CADVTLEGQVYPGKGGGSQEKNKTKQEKGKKKKEGVPKSRAKVVQEDNRAGNKREEL

SEQ ID NO: 3 - protein, 55 aa, Homo sapiens
CADVTLEGQVYPGKGGGSKEKNKTKQDKGKKKKEGDLKSRSSKEENRAGNKREDL

SEQ ID NO: 4 - protein, 54 aa, Mus musculus
CAEVTLEGQMYPGKGGGSKEKNKTKPEKAKKKEGDPKPRASKEDNRAGSRREDL

SEQ ID NO: 5 - protein, 54 aa, Mus musculus
CAEVTLEGQMYPGKGGGSKEKNKTKPEKAKKKEGDRKPRASKEDNRAGSRREDL

SEQ ID NO: 6 - protein, 55 aa, Pongo pygmaeus
CADVTLEGQVYPGKGGGSKEKNKTKQDKGKKKKEGDLKSRSSKEDNRARNKREDL

SEQ ID NO: 7 - protein, 54 aa, Rattus norvegicus
CAEVTLEGQMYPGKGGGSKEKNKTKPEKGKKKEGDPKPRASKEDNRAGSRREDL

SEQ ID NO: 8 - protein, 55 aa, Gallus gallus
CADVTLEGQVYPGKGADGSEKGRNKTKPEKAKKKKDAEKSKSSHEDNRANQTERG

SEQ ID NO: 9 - protein, 10 aa, Artificial
  Synthetic
KGGGSKEKNK

SEQ ID NO: 10 - protein, 10 aa, Artificial
  Synthetic
KGGGSQEKNK

SEQ ID NO: 11 - protein, 10 aa, Artificial
  Synthetic
KGGGSKEKNQ

SEQ ID NO: 12 - protein, 10 aa, Artificial
  Synthetic
KGGGSKERQL

SEQ ID NO: 13 - protein, 34 aa, Artificial
  Synthetic
KGGGSKEKNKTKQDKGKKKKEGDLKSRSSKEENR

SEQ ID NO: 14 - protein, 38 aa, Artificial
  Synthetic
KGGGSKEKNKTKQDKGKKKKEGDLKSRSSKEENRAGNK

SEQ ID NO: 15 - protein, 47 aa, Artificial
  Synthetic
QVYPGKGGGSKEKNKTKQDKGKKKKEGDLKSRSSKEENRAGNKREDL

SEQ ID NO: 16 - protein, 33 aa, Artificial
  Synthetic
KGGGSKEKNKTKPEKAKKKEGDPKPRASKEDNR

SEQ ID NO: 17 - protein, 33 aa, Artificial
  Synthetic
KGGGSKEKNKTKPEKAKKKEGDRKPRASKEDNR

SEQ ID NO: 18 - protein, 37 aa, Artificial
  Synthetic
SKEKNKTKPEKAKKKEGDPKPRASKEDNRAGSRREDL

SEQ ID NO: 19 - protein, 37 aa, Artificial
  Synthetic
SKEKNKTKPEKAKKKEGDRKPRASKEDNRAGSRREDL

SEQ ID NO: 20 - protein, 37 aa, Artificial
  Synthetic
KGGGSKEKNKTKPEKAKKKEGDPKPRASKEDNRAGSR

SEQ ID NO: 21 - protein, 37 aa, Artificial
  Synthetic
KGGGSKEKNKTKPEKAKKKEGDRKPRASKEDNRAGSR

SEQ ID NO: 22 - protein, 46 aa, Artificial
  Synthetic
QMYPGKGGGSKEKNKTKPEKAKKKEGDPKPRASKEDNRAGSRREDL

SEQ ID NO: 23 - protein, 18 aa, Artificial
  Synthetic
EGDPKPRASKEDNRAGSR

SEQ ID NO: 24 - protein, 18 aa, Artificial
  Synthetic
EGDRKPRASKEDNRAGSR

SEQ ID NO: 25 - protein, 18 aa, Artificial
  Synthetic
TKPEKAKKKEGDPKPRAS

SEQ ID NO: 26 - protein, 19 aa, Artificial
  Synthetic
KGGGSKEKNKTKPEKAKKK

SEQ ID NO: 27 - protein, 18 aa, Artificial
  Synthetic
TKPEKAKKKEGDRKPRAS

SEQ ID NO: 28 - protein, 9 aa, Artificial
  Synthetic
KEDNRAGSR

SEQ ID NO: 29 - protein, 10 aa, Artificial
  Synthetic
KEKNKTKPEK

SEQ ID NO: 30 - protein, 232 aa, Bos taurus
MAASGWARAAVIFLCACDLLLLLLLPPRAFATEGPAETPGEATPPPRKKKKDIRDYNDADMARLLEQWEKDDDIEEGDLPEHKRPSAPIDFSQIDPGKPESILKMTKKGKTLMMFVTVSGNPTEKETEEITSLWQGSLFNANYDVQRFIVGSDRAIFMLRDGGYAWEIKDFLVSQDRCADVTLEGQVYPGKGGGSKEKNQTKQEKGKKKKERDLKPRASKEDNRAGSKKEEL

SEQ ID NO: 31 - protein, 190 aa, Canis familiaris
MGSHVLVTRVIGAESCWRLGLHLKKDDDIEEGDLPEHKRPSAPIDFSQIDPGRPESILKMTKKGKTLMMFVTVSGSPTEKETEEITSLWQGSLFNANYDVQRFIVGSDRAIFMLRDGSYAWEIKDFLVSQDRCADVTLEGQVYPGKGGGSQEKNKTKQEKGKKKKEGVPKSRAAKVVQEDNRAGNKREEL

SEQ ID NO: 32 - protein, 234 aa, Homo sapiens
MAASRWARKAVVLLCASDLLLLLLLLPPPGSCAAEGSPGTPDESTPPPRKKKKDIRDYNDADMARLLEQWEKDDDIEEGDLPEHKRPSAPVDFSKIDPSKPESILKMTKKGKTLMMFVTVSGSPTEKETEEITSLWQGSLFNANYDVQRFIVGSDRAIFMLRDGSYAWEIKDFLVGQDRCADVTLEGQVYPGKGGGSKEKNKTKQDKGKKKKEGDLKSRSSKEENRAGNKREDL

SEQ ID NO: 33 - protein, 223 aa, Gallus gallus
MAAAARWAALGLALWLCAAAHAEEPEGKRRAGPAKKKDIRDYNDADMARLLEQWEKDDDIEEGDLPEHKRPPAPIDFSKIDPGKPESILKLTKKGKTLMMFVTVSGNPTEKETEEITSLWQGSLFNANYDVQRFIVGSNRAIFMLRDGGYAWEIKDFLISQERCADVTLEGQVYPGKGADGSEKGRNKTKPEKAKKKKDAEKSKSSHEDNRANQTERGSMTDT

SEQ ID NO: 34 - protein, 224 aa, Mus musculus
MAASRWLRAVLLFLCASDLLLLPPPNAYAADTPGEATPPPRKKKDIRDYNDADMARLLEQWEKDDDIEEGDLPEHKRPSAPIDFSKLDPGKPESILKMTKKGKTLMMFVTVSGNPTEKETEEITSLWQGSLFNANYDVQRFIVGSDRAIFMLRDGSYAWEIKDFLVSQDRCAEVTLEGQMYPGKGGGSKEKNKTKPEKAKKKEGDRKPRASKEDNRAGSRREDL

SEQ ID NO: 35 - protein, 235 aa, Pan troglodytes
KMAASRWARKAVVLLCASDLLLLLLLLPPPGSCAAEGSPGTPDESTPPPRKKKKDIRDYNDADMARLLEQWEKDDDIEEGDLPEHKRPSAPVDFSKIDPSKPESILKMTKKGKTLMMFVTVSGSPTEKETEEITSLWQGSLFNANYDVQRFIVGSDRAIFMLRDGSYAWEIKDFLVGQDRCADVTLEGQVYPGKGGGSKEKNKTKQDKGKKKKEGDLKSRSSKEENRAGNKREDL

SEQ ID NO: 36 - protein, 234 aa, Pongo pygmaeus
MAASSWARKAVVVLCASDLLLLLLLLPPPGSCAAEASPGTPDESTPPPRKKKKDIRDYNDADMARLLEQWEKDDDIEEGDLPEHKRPSAPVDFSKIDPSKPESILKMTKKGKTLMMFVTVSGSPTEKETEEITSLWQGSLFNANYDVQRFIVGSDRAIFMLRDGNYAWEIKDFLVGQDRCADVTLEGQVYPGKGGGSKEKNKTKQDKGKKKKEGDLKSRSSKEDNRARNKREDL

SEQ ID NO: 37 - protein, 224 aa, Rattus norvegicus
MAASSWLRAVLLFLCASDLLLLSPPEAYATDTPGEAITPPRKKKDIRDYNDADMARLLEQWEKDDDIEEGDLPEHKRPSAPIDFSKLDPGKPESILKMTKKGKTLMMFVTISGNPTEKETEEITSLWQGSLFNANYDVQRFIVGSDRAIFMLRDGSYAWEIKDFLVNQDRCAEVTLEGQMYPGKGGGSKEKNKTKPEKGKKKEGDPKPRASKEDNRAGSRREDL

SEQ ID NO: 38 - protein, 158 aa, Xenopus laevis
MGRSRSRSPERRRERRRSRSASRERERRRRERSRSRERRRSRSRSPHRRRSRSPRRHRSSSISPSRLKDRRDDDKKEPKESKGGGSKERQLAAEDLEGKTEEEIEMMKLMGFASFDSSKGKKTDGSVNAYAINVSQKRKYRQYMNRKGGFNRPLDFVA

(19) United States
(12) Patent Application Publication
(10) Pub. No.: US 2008/0227698 A1
(43) Pub. Date: Sep. 18, 2008

(54) OLIGOPEPTIDES FOR TREATMENT OF OSTEOPOROSIS AND OTHER BONE DISEASES AND METHODS THEREOF

(75) Inventors: Guojun Bu, Chesterfield, MO (US); Yonghe Li, Birmingham, AL (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Washington University

(21) Appl. No.: 11/557,292

(22) Filed: Nov. 7, 2006

Related U.S. Application Data
(60) Provisional application No. 60/734,556, filed on Nov. 8, 2005.

(51) Int. Cl. A61K 38/16; C07K 7/06; C07K 14/00; C07K 7/08; C12N 15/63; A61P 19/08; A61P 19/10; A61K 31/7052; C12N 5/00; A61K 38/08; A61K 38/10

(52) U.S. Cl. 514/12; 530/328; 530/324; 530/326; 435/320.1; 435/325; 514/15; 514/13; 514/44

(57) ABSTRACT

Oligopeptides which can be used to treat bone disease such as osteoporosis are disclosed. Further disclosed are methods of treating bone disease such as osteoporosis. These methods include administration of a polypeptide encoded by the Mesd gene, or an oligopeptide comprising a contiguous subsequence of a Mesd polypeptide.

### CROSS-REFERENCE TO RELATED APPLICATIONS

This application claims priority from U.S. Provisional Application Ser. No. 60/734,556 filed on Nov. 8, 2005, which is incorporated herein by reference in its entirety.

### STATEMENT REGARDING FEDERALLY SPONSORED RESEARCH OR DEVELOPMENT

This invention was made in part with Government support under R01-CA100520 from the National Institutes of Health. The Government has certain rights in the invention.

### INCORPORATION-BY-REFERENCE OF MATERIAL SUBMITTED ON A COMPACT DISC

The Sequence Listing, which is a part of the present disclosure, includes a computer readable form and a written sequence listing comprising nucleotide and/or amino acid sequences of the present invention. The sequence listing information recorded in computer readable form is identical to the written sequence listing. The subject matter of the Sequence Listing is incorporated herein by reference in its entirety.

### BACKGROUND

Osteoporosis and other diseases involving altered bone mass and/or bone degeneration present significant health problems. Osteoporosis is a systemic skeletal disease characterized by low bone mass and microarchitectural deterioration of bone tissue, with a consequent increase in bone fragility and a susceptibility to fracture. Current methods of treatment of osteoporosis and other bone disorders can be expensive, unavailable, or ineffective for some subjects. There is, therefore, an unmet need for new modalities for treatment.

Canonical Wnt signaling is critical for postnatal bone accrual (Ferrari, S. L., Curr. Opin. Lipidol. 16: 207-214, 2005; Westendorf, J. J., Gene 341: 19-39, 2004). In the canonical Wnt-signaling pathway, a Wnt growth factor must bind to both its receptor and either an LRP5 or an LRP6 co-receptor to initiate signaling (He, X., Development 131: 1663-1677, 2004).

Signaling by the low-density lipoprotein receptor (LDLR)-related protein-5 (LRP5) and (LDLR)-related protein-6 (LRP6), which are both members of the LDLR family (Herz, J., Annu. Rev. Biochem. 71, 405-434, 2002; Schneider, W. J., Cell Mol. Life Sci. 60, 892-903, 2003; He, X., Development 131: 1663-1677, 2004), is subject to inhibition by the extracellular Wnt signaling molecule DKK1, a member of the dickkopf gene family prevalent in bone. Substantial genetic data implicate LRP5 as a regulator of bone density (Ferrari, S. L., Curr. Opin. Lipidol. 16: 207-214, 2005; Westendorf, J. J., Gene 341: 19-39, 2004): LRP5 loss-of-function mutations cause the human autosomal recessive disorder osteoporosis-pseudoglioma syndrome (Gong, Y., Cell 107: 513-523, 2001), whereas gain-of-function mutations in LRP5 result in the autosomal-dominant high-bone-mass trait in humans (Boyden, L. M., N. Engl. J. Med. 346: 1513-1521, 2002; Little, R. D., Am. J. Hum. Genet. 70: 11-19, 2002). The extracellular molecule dickkopf (Dkk1) has the ability to interact with LRP5 and another transmembrane protein, Kremen, triggering internalization and inactivation of LRP5 (Mao, B., Nature 417: 664-667, 2002). The production of DKK1, an inhibitor of osteoblast differentiation, by myeloma cells is associated with the presence of lytic bone lesions in subjects with multiple myeloma (Tian, E., N. Engl. J. Med. 349: 2483-94, 2003).

### SUMMARY

In view of the unmet need for new treatments for diseases and disorders involving altered bone mass, including degenerative bone diseases such as osteoporosis, the present inventors have developed oligopeptides which can prevent, slow or reverse bone degeneration, or promote bone growth when applied to osteoblasts in vitro or in vivo. The inventors have also developed methods of treating bone disorders and promoting bone growth. These methods use the oligopeptides as well as a full-length polypeptide encoded by a mesd gene.

In some configurations, the inventors have developed oligopeptides which comprise contiguous subsequences of a polypeptide encoded by a mesd gene. An oligopeptide of these configurations can comprise a contiguous amino acid sequence of from about 10 contiguous amino acids in length up to about 70 contiguous amino acids in length. In some aspects, an oligopeptide can be from about 30 contiguous amino acids in length up to about 67 contiguous amino acids in length. In various aspects, an oligopeptide can comprise a contiguous amino acid sequence selected from

(SEQ ID NO: 1)

CADVTLEGQVYPGKGGGSKEKNQTKQEKGKKKKERDLKPRASKEDNRAGS
KKEEL,

(SEQ ID NO: 2)

CADVTLEGQVYPGKGGGSQEKNKTKQEKGKKKKEGVPKSRAKVVQEDNRA
GNKREEL,

(SEQ ID NO: 3)

CADVTLEGQVYPGKGGGSKEKNKTKQDKGKKKKEGDLKSRSSKEENRAGN
KREDL,

(SEQ ID NO: 4)

CAEVTLEGQMYPGKGGGSKEKNKTKPEKAKKKEGDPKPRASKEDNRAGSR
REDL,

(SEQ ID NO: 5)

CAEVTLEGQMYPGKGGGSKEKNKTKPEKAKKKEGDRKPRASKEDNRAGSR
REDL,

(SEQ ID NO: 6)

CADVTLEGQVYPGKGGGSKEKNKTKQDKGKKKKEGDLKSRSSKEDNRARN
KREDL,

(SEQ ID NO: 7)

CAEVTLEGQMYPGKGGGSKEKNKTKPEKGKKKEGDPKPRASKEDNRAGSR
REDL,

(SEQ ID NO: 8)

CADVTLEGQVYPGKGADGSEKGRNKTKPEKAKKKKDAEKSKSSHEDNRAN
QTERG,

(SEQ ID NO: 9)

KGGGSKEKNK,

(SEQ ID NO: 10)

KGGGSQEKNK,

(SEQ ID NO: 11)

KGGGSKEKNQ,

(SEQ ID NO: 12)

KGGGSKERQL

(SEQ ID NO: 13)

KGGGSKEKNKTKQDKGKKKKEGDLKSRSSKEENR,

(SEQ ID NO: 14)

KGGGSKEKNKTKQDKGKKKKEGDLKSRSSKEENRAGNK,

(SEQ ID NO: 15)

QVYPGKGGGSKEKNKTKQDKGKKKKEGDLKSRSSKEENRAGNKREDL,

(SEQ ID NO: 16)

KGGGSKEKNKTKPEKAKKKEGDPKPRASKEDNR,

(SEQ ID NO: 17)

KGGGSKEKNKTKPEKAKKKEGDRKPRASKEDNR,

(SEQ ID NO: 18)

SKEKNKTKPEKAKKKEGDPKPRASKEDNRAGSRREDL,

(SEQ ID NO: 19)

SKEKNKTKPEKAKKKEGDRKPRASKEDNRAGSRREDL,

(SEQ ID NO: 20)

KGGGSKEKNKTKPEKAKKKEGDPKPRASKEDNRAGSR,

(SEQ ID NO: 21)

KGGGSKEKNKTKPEKAKKKEGDRKPRASKEDNRAGSR,

(SEQ ID NO: 22)

QMYPGKGGGSKEKNKTKPEKAKKKEGDPKPRASKEDNRAGSRREDL,

(SEQ ID NO: 23)

EGDPKPRASKEDNRAGSR,

(SEQ ID NO: 24)

EGDRKPRASKEDNRAGSR,

(SEQ ID NO: 25)

TKPEKAKKKEGDPKPRAS,

(SEQ ID NO: 26)

KGGGSKEKNKTKPEKAKKK,

(SEQ ID NO: 27)

TKPEKAKKKEGDRKPRAS,

(SEQ ID NO: 28)

KEDNRAGSR
and

(SEQ ID NO: 29)

KEKNKTKPEK.

In some aspects, an oligopeptide can comprise a sequence selected from the group consisting of

(SEQ ID NO: 14)

KGGGSKEKNKTKQDKGKKKKEGDLKSRSSKEENRAGNK

(SEQ ID NO: 20)

KGGGSKEKNKTKPEKAKKKEGDPKPRASKEDNRAGSR,

(SEQ ID NO: 21)

KGGGSKEKNKTKPEKAKKKEGDRKPRASKEDNRAGSR
and

(SEQ ID NO: 22)

QMYPGKGGGSKEKNKTKPEKAKKKEGDPKPRASKEDNRAGSRREDL.

In some aspects, an oligopeptide can consist essentially of a sequence selected from the group consisting of

(SEQ ID NO: 14)

KGGGSKEKNKTKQDKGKKKKEGDLKSRSSKEENRAGNK

(SEQ ID NO: 20)

KGGGSKEKNKTKPEKAKKKEGDPKPRASKEDNRAGSR,

(SEQ ID NO: 21)

KGGGSKEKNKTKPEKAKKKEGDRKPRASKEDNRAGSR
and

(SEQ ID NO: 22)

QMYPGKGGGSKEKNKTKPEKAKKKEGDPKPRASKEDNRAGSRREDL.

In some aspects, an oligopeptide can consist of a sequence selected from the group consisting of

(SEQ ID NO: 14)

KGGGSKEKNKTKQDKGKKKKEGDLKSRSSKEENRAGNK

(SEQ ID NO: 20)

KGGGSKEKNKTKPEKAKKKEGDPKPRASKEDNRAGSR,

(SEQ ID NO: 21)

KGGGSKEKNKTKPEKAKKKEGDRKPRASKEDNRAGSR
and

(SEQ ID NO: 22)

QMYPGKGGGSKEKNKTKPEKAKKKEGDPKPRASKEDNRAGSRREDL.

In some aspects, an oligopeptide of the present teachings which is at least about 10 contiguous amino acids in length up to about 70 contiguous amino acids in length can include conservative amino acid substitutions at one or more positions, as compared to an oligopeptide of a sequence set forth as SEQ ID NO: 1 through SEQ ID NO: 29. In various aspects, an oligopeptide of the present teachings can exhibit a biochemical property of antagonizing binding of an LRP5 inhibitor, such as a DKK1 polypeptide, to an LRP5 receptor, as described below.

In related aspects, the present teachings also include oligopeptides having at least about 70% sequence identity, at least about 75% sequence identity, at least about 80% sequence identity, at least about 85% sequence identity, and least about 90% sequence identity, at least about 95% sequence identity, at least about 96% sequence identity, at least about 97% sequence identity, at least about 98% sequence identity, or at least about 99% sequence identity with a homologous sequence from a polypeptide set forth as SEQ ID NO: 1 through SEQ ID NO: 29. In various configurations, an oligopeptide can be a substantially pure oligopeptide or an isolated oligopeptide, including a substantially pure or isolated full-length Mesd polypeptide or a portion thereof such as an oligopeptide having a sequence set forth in SEQ ID NO: 1 through SEQ ID NO: 29, or an oligopeptide having conservative substitutions with respect to a sequence of a full-length Mesd polypeptide (SEQ ID NO: 30 through SEQ ID NO: 38) or an oligopeptide set forth as SEQ ID NO: 1 through SEQ ID NO: 29.

In various aspects, the sequence of a Mesd polypeptide or a portion thereof can be that of a polypeptide or a portion thereof comprising at least about 10 contiguous amino acids and encoded by a mesd gene from any animal, including, in non-limiting example, a vertebrate such as a fish, an amphibian, a reptile such as Xenopus laevis, a bird such as Gallus gallus or a mammal such as a human or rodent such as Mus musculus, provided the sequence shares at least about 70% sequence identity with a Mesd polypeptide or at least one sequence set forth as SEQ ID NO: 1 through SEQ ID NO: 29. In various aspects, the sequence can share at least about 70% sequence identity, at least about 75% sequence identity, at least about 80% sequence identity, at least about 85% sequence identity, and least about 90% sequence identity, at least about 95% sequence identity, at least about 96% sequence identity, at least about 97% sequence identity, at least about 98% sequence identity, or at least about 99% sequence identity with a Mesd polypeptide (e.g., SEQ ID NO: 30-38) or at least one sequence set forth as SEQ ID NO: 1 through SEQ ID NO: 29. In various aspects, a Mesd polypeptide or peptide can be at least about 20 contiguous amino acids, at least about 30 contiguous amino acids, or at least 37 contiguous amino acids in length. In various aspects, the polypeptide or oligopeptide antagonizes binding of DKK1 to LRP5 when in contact with LRP5, and/or relieves DKK1-mediated inhibition of Wnt signaling.

In other configurations, the present teachings include methods of treatment of degenerative bone disease such as osteoporosis. A method of these configurations comprises administering to a subject in need of therapy, such as a human subject diagnosed with osteoporosis, a therapeutically effective amount of a Mesd polypeptide (e.g., SEQ ID NO: 30 through SEQ ID NO: 38); an oligopeptide which is at least about 10 contiguous amino acids in length up to about 70 contiguous amino acids in length and comprises a sequence set forth in SEQ ID NO: 1 through SEQ ID NO: 29, or a sequence comprising at least 10 contiguous amino acids in length up to about 70 contiguous amino acids in length and sharing at least about 70% sequence identity, at least about 75% sequence identity, at least about 80% sequence identity, at least about 85% sequence identity, and least about 90% sequence identity, at least about 95% sequence identity, at least about 96% sequence identity, at least about 97% sequence identity, at least about 98% sequence identity, or at least about 99% sequence identity with a Mesd polypeptide (e.g., SEQ ID NO: 30 through SEQ ID NO: 38) or at least one sequence set forth as SEQ ID NO: 1 through SEQ ID NO: 29. In related aspects, degenerative bone disease can be treated by administering to a subject a full length Mesd polypeptide, such as SEQ ID NOS: 30-38, including a mammalian Mesd polypeptide such as a human or murine Mesd polypeptide. In some configurations, degenerative bone disease can be treated by administering to a subject a polypeptide sharing at least about 70% sequence identity, at least about 75% sequence identity, at least about 80% sequence identity, at least about 85% sequence identity, and least about 90% sequence identity, at least about 95% sequence identity, at least about 96% sequence identity, at least about 97% sequence identity, at least about 98% sequence identity, or at least about 99% sequence identity with full length Mesd polypeptide, such as SEQ ID NO: 30-38, including a mammalian Mesd polypeptide such as a human or murine Mesd polypeptide.

In various other aspects, the inventors have developed vectors comprising a promoter operably linked to a nucleic acid sequence encoding a Mesd polypeptide, or an oligopeptide which comprises a sequence from a polypeptide encoded by the mesd gene as described herein. In various aspects, a vector can be a plasmid or a virus, and a promoter can be a eukaryotic promoter or a prokaryotic promoter. These vectors can be used to produce an oligopeptide ex vivo, or can be used therapeutically, such as by administering a vector described in the present teachings, or by administering, to a subject in need of treatment, cells comprising the vector and which produce a full-length Mesd polypeptide or a Mesd oligopeptide. In such configurations, a vector can comprise, in addition to a promoter and a nucleic acid encoding an Mesd polypeptide or oligopeptide, sequences linked to those encoding the polypeptide or oligopeptide and promote export or secretion from a cell.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 illustrates that Mesd binds to mature LRP6 at the cell surface with high affinity.

FIG. 2 illustrates that Mesd binds to mature LRP5 and LRP6 but not significantly to other members of the LDLR family.

FIG. 3 illustrates that the C-terminal region of Mesd is required for interaction with LRP6.

FIG. 4 illustrates that the C-terminal region of Mesd is necessary and sufficient for LRP6 binding.

FIG. 5 illustrates that the carboxy-terminal region of Mesd is required for LRP6 folding.

FIG. 6 illustrates that LRP6 is not a constitutively active endocytosis receptor.

FIG. 7 illustrates that LRP6 exhibits a limited level of Mesd degradation.

FIG. 8 illustrates that the 39 kDa specialized molecular chaperone receptor-associated protein RAP binds to LRP6 and partially competes for Mesd binding.

FIG. 9 illustrates that Mesd inhibits DKK1 binding to LRP6.

FIG. 10 illustrates that Mesd polypeptide or a Mesd oligopeptide can both bind LRP5 and inhibit binding of DKK1 to either LRP5 or LRP6.

FIG. 11 illustrates rescue of alkaline phosphatase activity in DKK1-inhibited ST-2 osteoblast-derived cells.

FIG. 12 illustrates Wnt signaling, its inhibition by DKK1, and partial reversal of the inhibition by a Mesd protein or oligopeptide.

### DETAILED DESCRIPTION OF THE PRESENTLY PREFERRED EMBODIMENTS

The present inventors disclose substantially pure oligopeptides which can be used to treat bone disease, including degenerative bone disease. “Bone disease,” as used herein, refers to disorders and diseases of bone, as well as changes associated with aging. In various configurations, a bone disease can be osteoporosis. The methods disclosed herein can be applied to both humans and animals, including, without limitation, companion animals, agricultural animals and laboratory animals, and can be used for preventing or slowing osteoporosis.

Without being limited by theory, the inventors presume that signaling by the low-density lipoprotein receptor (LDLR)-related protein-5 (LRP5) and (LDLR)-related protein-6 (LRP6) which are both members of the LDLR family (Herz, J., Annu. Rev. Biochem. 71, 405-434, 2002; Schneider, W. J., Cell Mol. Life Sci. 60, 892-903, 2003; He, X., Development 131: 1663-1677, 2004), is subject to inhibition by the extracellular Wnt signalling molecule DKK1, a member of the dickkopf gene family. By binding to LRP5/LRP6, DKK1 is believed to disrupt the binding of LRP5 to the Wnt/Frizzled ligand-receptor complex, which leads to inhibition of Wnt/β-catenin signaling (Semenov, M. V., Current Biology 11, 951-961, 2001; Rawadi, G., Expert Opin. Ther. Targets 9: 1063-1077, 2005). Recently, a novel specialized chaperone for members of the LDLR family, termed Mesd (mesoderm development) in mouse and Boca in Drosophila has been identified (Culi, J., Cell 112: 343-354, 2003; Hsieh, J. C., Cell 112: 355-367, 2003). This new chaperone was discovered due to its requirement for the folding of LRP5/LRP6, co-receptors for the Wnt/Wg signaling pathway. However, the present inventors find that Mesd not only mediates folding of LRP5 and LRP6, it also is capable of binding mature LRP5 or LRP6 at the cell surface, and antagonizes binding of ligand such as DKK1. In addition, the present inventors find the ligand-binding antagonizing activity is found in oligopeptides comprising subsequences of Mesd from the carboxy-terminal region of the Mesd polypeptide. The present inventors have found that Mesd polypeptides, as well as oligopeptides comprising Mesd carboxy-terminal sequences, stimulate bone formation when contacted with osteoblasts. Again, without being limited by theory, the inventors attribute this effect of Mesd or a Mesd oligopeptide to interference with DKK1 binding to LRP5. Furthermore, the inventors believe, without being limited by theory, that because interferening with DKK1 binding to LRP5 using Mesd or a Mesd oligopeptide stimulates bone formation or slows osteoporosis by relieving an inhibition, the methods avoid overstimulating target cells (osteoblasts) with excessive Wnt signalling.

Oligopeptides of the present teachings comprise from about 10 contiguous amino acids up to about 70 contiguous amino acids, wherein the contiguous amino acids comprise an amino acid sequence selected from the group consisting of SEQ ID NO: 9, SEQ ID NO: 10, SEQ ID NO: 11, and SEQ ID NO: 12. Such sequences are generally 10 amino acids in length and may be in various positions within the larger oligopeptide, or may consist, or consist essentially, of the oligopeptide.

The oligopeptide can be, for example, about 10 contiguous amino acids, about 15 contiguous amino acids, about 20 contiguous amino acids, about 25 contiguous amino acids, about 30 contiguous amino acids, about 35 contiguous amino acids, about 40 contiguous amino acids, about 45 contiguous amino acids, about 50 contiguous amino acids, about 55 contiguous amino acids, about 60 contiguous amino acids, about 65 contiguous amino acids, or about 70 contiguous amino acids. In various configurations, a substantially pure oligopeptide of the present teachings can comprise at least about 20 or at least about 30 contiguous amino acids, up to about 67 amino acids. Exemplary sequences of the present teachings are set forth herein in Table 1, which presents sequences of from 54 to 58 contiguous amino acids (SEQ ID NOS: 1-8); of from 33 to 47 contiguous amino acids (SEQ ID NOS: 13-22); of from 18 to 19 contiguous amino acids (SEQ ID NOS: 23-27); and of from 9 to 10 contiguous amino acids (SEQ ID NOS: 9-12, 28-29). The oligopeptides of the invention can comprise such sequences. Alternatively, the oligopeptides of the invention can consist, or consist essentially, of such sequences. That is to say, the oligopeptides of the invention may be the actual sequence set forth or may include such sequence.

Various sequences presented herein represent subsequences from a Mesd polypeptide encoded by a mesd gene comprised by the genome of a variety of species such as, without limitation, human, mouse, dog, cow, chimpanzee, orangutan, chicken, and rat. As used herein, the term “oligopeptide” generally refers to a molecule comprising at least two amino acids joined by peptide bonds, and the term “polypeptide” generally refers to a molecule comprising a full-length amino acid sequence as encoded by a gene, an mRNA, or a cDNA.

An oligopeptide and/or polypeptide of the present teachings can be synthesized using standard techniques well known to skilled artisans, such as, in non-limiting example Merrifield solid phase synthesis, or molecular cloning methods, including, in non-limiting example, synthesizing an oligonucleotide encoding an oligopeptide and inserting the oligonucleotide into a vector, or subcloning a portion of a cDNA into a vector using restriction enzyme digestion, ligation with a ligase, and/or polymerase chain reaction techniques. A vector comprising an oligonucleotide encoding an oligopeptide can in inserted into a cell by transfection or transformation, and expressed in the cell using methods well known to skilled artisans. Oligopeptides can be isolated and/or purified by standard techniques well known to skilled artisans.

Accordingly, in some configurations of the present teachings, an oligopeptide can be from about 10 amino acids in length up to about 70 amino acids in length. The sequence can comprise, consist essentially, or consist of any sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, and SEQ ID NO: 29. Furthermore, a sequence of an oligopeptide can be a sequence sharing at least about 70% sequence identity, at least about 75% sequence identity, at least about 80% sequence identity, at least about 85% sequence identity, and least about 90% sequence identity, at least about 95% sequence identity, at least about 96% sequence identity, at least about 97% sequence identity, at least about 98% sequence identity, or at least about 99% sequence identity with at least one sequence of SEQ ID NO: 1 through SEQ ID NO: 29 and has the biochemical property of antagonizing, inhibiting, or blocking binding of a mature cell surface protein LRP5 with an extracellular ligand such as DKK1, when the oligopeptide is contacted with an LRP5 such as an LRP5 comprised by a cell membrane.

Furthermore, a sequence of an oligopeptide can be a sequence sharing at least about 70% sequence identity, at least about 75% sequence identity, at least about 80% sequence identity, at least about 85% sequence identity, and least about 90% sequence identity, at least about 95% sequence identity, at least about 96% sequence identity, at least about 97% sequence identity, at least about 98% sequence identity, or at least about 99% sequence identity with at least one sequence of SEQ ID NO: 1 through SEQ ID NO: 29 and has the biochemical property of enhancing Wnt signaling when such signaling is inhibited by an extracellular ligand such as DKK1, such as when a cell subjected to Wnt signaling is contacted with the oligopeptide.

In various configurations, conservative substitutions can be made in oligopeptide sequences, for example substitution of a hydrophobic amino acid such as valine with a different hydrophobic amino acid such as isoleucine. Methods for identifying and selecting conservative substitutions for amino acids are well known to skilled artisans (see, e.g., Pearson, W. R., Methods Enzymol. 266: 227-258,1996).

TABLE 1

| | | | |
|---|---|---|---|
| SEQ | | | |
| ID | | | Acc. |
| NO: | Amino Acid Sequence | Species | No. |

| | | | |
|---|---|---|---|
| 1 | CADVTLEGQVYPGKGGGSKEKNQTKQEKGKKKKERDLKPRASKEDNRAG | Bos taurus | |
| | SKKEEL | (cow) | |
| 2 | CADVTLEGQVYPGKGGGSQEKNKTKQEKGKKKKEGVPKSRAKVVQEDNR | Canis | |
| | AGNKREEL | familiaris | |
| | | (dog) | |
| 3 | CADVTLEGQVYPGKGGGSKEKNKTKQDKGKKKKEGDLKSRSSKEENRAG | Homo | |
| | NKREDL | sapiens | |
| | | (human) | |
| 4 | CAEVTLEGQMYPGKGGGSKEKNKTKPEKAKKKEGDPKPRASKEDNRAGS | Mus | |
| | RREDL | musculus | |
| | | (house | |
| | | mouse) | |
| 5 | CAEVTLEGQMYPGKGGGSKEKNKTKPEKAKKKEGDRKPRASKEDNRAGS | Mus | |
| | RREDL | musculus | |
| | | (house | |
| | | mouse) | |
| 6 | CADVTLEGQVYPGKGGGSKEKNKTKQDKGKKKKEGDLKSRSSKEDNRAR | Pongo | |
| | NKREDL | pygmaeus | |
| | | (orangutan) | |
| 7 | CAEVTLEGQMYPGKGGGSKEKNKTKPEKGKKKEGDPKPRASKEDNRAGS | Rattus | |
| | RREDL | norvegicus | |
| | | (Norway | |
| | | rat) | |
| 8 | CADVTLEGQVYPGKGADGSEKGRNKTKPEKAKKKKDAEKSKSSHEDNRA | Gallus | |
| | NQTERG | gallus | |
| | | (chicken) | |
| 9 | KGGGSKEKNK | Homo | |
| | | sapiens | |
| | | (human) | |
| 10 | KGGGSQEKNK | Canis | |
| | | familiaris | |
| | | (dog) | |
| 11 | KGGGSKEKNQ | Bos taurus | |
| | | (cow) | |
| 12 | KGGGSKERQL | Xenopus | |
| | | laevis | |
| | | (African | |
| | | clawed | |
| | | frog) | |
| 13 | KGGGSKEKNKTKQDKGKKKKEGDLKSRSSKEENR | Homo | |
| | | sapiens | |
| | | (human) | |
| 14 | KGGGSKEKNKTKQDKGKKKKEGDLKSRSSKEENRAGNK | Homo | |
| | | sapiens | |
| | | (human) | |
| 15 | QVYPGKGGGSKEKNKTKQDKGKKKKEGDLKSRSSKEENRAGNKREDL | Homo | |
| | | sapiens | |
| | | (human) | |
| 16 | KGGGSKEKNKTKPEKAKKKEGDPKPRASKEDNR | Mus | |
| | | musculus | |
| | | (house | |
| | | mouse) | |
| 17 | KGGGSKEKNKTKPEKAKKKEGDRKPRASKEDNR | Mus | |
| | | musculus | |
| | | (house | |
| | | mouse) | |
| 18 | SKEKNKTKPEKAKKKEGDPKPRASKEDNRAGSRREDL | Mus | |
| | | musculus | |
| | | (house | |
| | | mouse) | |
| 19 | SKEKNKTKPEKAKKKEGDRKPRASKEDNRAGSRREDL | Mus | |
| | | musculus | |
| | | (house | |
| | | mouse) | |
| 20 | KGGGSKEKNKTKPEKAKKKEGDPKPRASKEDNRAGSR | Mus | |
| | | musculus | |
| | | (house | |
| | | mouse) | |
| 21 | KGGGSKEKNKTKPEKAKKKEGDRKPRASKEDNRAGSR | Mus | |
| | | musculus | |
| | | (house | |
| | | mouse) | |
| 22 | QMYPGKGGGSKEKNKTKPEKAKKKEGDPKPRASKEDNRAGSRREDL | Mus | |
| | | musculus | |
| | | (house | |
| | | mouse) | |
| 23 | EGDPKPRASKEDNRAGSR | Mus | |
| | | musculus | |
| | | (house | |
| | | mouse) or | |
| | | Rattus | |
| | | norvegicus | |
| | | (Norway | |
| | | rat) | |
| 24 | EGDRKPRASKEDNRAGSR | Mus | |
| | | musculus | |
| | | (house | |
| | | mouse) | |
| 25 | TKPEKAKKKEGDPKPRAS | Mus | |
| | | musculus | |
| | | (house | |
| | | mouse) | |
| 26 | KGGGSKEKNKTKPEKAKKK | Mus | |
| | | musculus | |
| | | (house | |
| | | mouse) | |
| 27 | TKPEKAKKKEGDRKPRAS | Mus | |
| | | musculus | |
| | | (house | |
| | | mouse) | |
| 28 | KEDNRAGSR | Mus | |
| | | musculus | |
| | | (house | |
| | | mouse) | |
| 29 | KEKNKTKPEK | Mus | |
| | | musculus | |
| | | (house | |
| | | mouse) | |
| 30 | MAASGWARAAVIFLCACDLLLLLLLPPRAFATEGPAETPGEATPPPRKK | Bos taurus | NM_001034469.1 |
| | KKDIRDYNDADMARLLEQWEKDDDIEEGDLPEHKRPSAPIDFSQIDPGK | (cow) | |
| | PESILKMTKKGKTLMMFVTVSGNPTEKETEEITSLWQGSLFNANYDVQR | | |
| | FIVGSDRAIFMLRDGGYAWEIKDFLVSQDRCADVTLEGQVYPGKGGGSK | | |
| | EKNQTKQEKGKKKKERDLKPRASKEDNRAGSKKEEL | | |
| 31 | MGSHVLVTRVIGAESCWRLGLHLKKDDDIEEGDLPEHKRPSAPIDFSQI | Canis | XM_545883.2 |
| | DPGRPESILKMTKKGKTLMMFVTVSGSPTEKETEEITSLWQGSLFNANY | familiaris | |
| | DVQRFIVGSDRAIFMLRDGSYAWEIKDFLVSQDRCADVTLEGQVYPGKG | (dog) | |
| | GGSQEKNKTKQEKGKKKKEGVPKSRAAKVVQEDNRAGNKREEL | | |
| 32 | MAASRWARKAVVLLCASDLLLLLLLLPPPGSCAAEGSPGTPDESTPPPR | Homo | NM_015154 |
| | KKKKDIRDYNDADMARLLEQWEKDDDIEEGDLPEHKRPSAPVDFSKIDP | sapiens | |
| | SKPESILKMTKKGKTLMMFVTVSGSPTEKETEEITSLWQGSLFNANYDV | (human) | |
| | QRFIVGSDRAIFMLRDGSYAWEIKDFLVGQDRCADVTLEGQVYPGKGGG | | |
| | SKEKNKTKQDKGKKKKEGDLKSRSSKEENRAGNKREDL | | |
| 33 | MAAAARWAALGLALWLCAAAHAEEPEGKRRAGPAKKKDIRDYNDADMAR | Gallus | NM_001030551 |
| | LLEQWEKDDDIEEGDLPEHKRPPAPIDFSKIDPGKPESILKLTKKGKTL | gallus | |
| | MMFVTVSGNPTEKETEEITSLWQGSLFNANYDVQRFIVGSNRAIFMLRD | (chicken) | |
| | GGYAWEIKDFLISQERCADVTLEGQVYPGKGADGSEKGRNKTKPEKAKK | | |
| | KKDAEKSKSSHEDNRANQTERGSMTDT | | |
| 34 | MAASRWLRAVLLFLCASDLLLLPPPNAYAADTPGEATPPPRKKKDIRDY | Mus | NM_023403 |
| | NDADMARLLEQWEKDDDIEEGDLPEHKRPSAPIDFSKLDPGKPESILKM | musculus | |
| | TKKGKTLMMFVTVSGNPTEKETEEITSLWQGSLFNANYDVQRFIVGSDR | (house | |
| | AIFMLRDGSYAWEIKDFLVSQDRCAEVTLEGQMYPGKGGGSKEKNKTKP | mouse) | |
| | EKAKKKEGDRKPRASKEDNRAGSRREDL | | |
| 35 | MAASRWARKAVVLLCASDLLLLLLLLPPPGSCAAEGSPGTPDESTPPPR | Pan | XM_510542.1 |
| | KKKKDIRDYNDADMARLLEQWEKDDDIEEGDLPEHKRPSAPVDFSKIDP | troglodytes | |
| | SKPESILKMTKKGKTLMMFVTVSGSPTEKETEEITSLWQGSLFNANYDV | (chimpanzee) | |
| | QRFIVGSDRAIFMLRDGSYAWEIKDFLVGQDRCADVTLEGQVYPGKGGG | | |
| | SKEKNKTKQDKGKKKKEGDLKSRSSKEENRAGNKREDL | | |
| 36 | MAASSWARKAVVVLCASDLLLLLLLLPPPGSCAAEASPGTPDESTPPPR | Pongo | CR860539 |
| | KKKKDIRDYNDADMARLLEQWEKDDDIEEGDLPEHKRPSAPVDFSKIDP | pygmaeus | |
| | SKPESILKMTKKGKTLMMFVTVSGSPTEKETEEITSLWQGSLFNANYDV | (orangutan) | |
| | QRFIVGSDRAIFMLRDGNYAWEIKDFLVGQDRCADVTLEGQVYPGKGGG | | |
| | SKEKNKTKQDKGKKKKEGDLKSRSSKEDNRARNKREDL | | |
| 37 | MAASSWLRAVLLFLCASDLLLLSPPEAYATDTPGEAITPPRKKKDIRDY | Rattus | NM_001008345 |
| | NDADMARLLEQWEKDDDIEEGDLPEHKRPSAPIDFSKLDPGKPESILKM | norvegicus | |
| | TKKGKTLMMFVTISGNPTEKETEEITSLWQGSLFNANYDVQRFIVGSDR | (Norway | |
| | AIFMLRDGSYAWEIKDFLVNQDRCAEVTLEGQMYPGKGGGSKEKNKTKP | rat) | |
| | EKGKKKEGDPKPRASKEDNRAGSRREDL | | |
| 38 | MGRSRSRSPERRRERRRSRSASRERERRRRERSRSRERRRSRSRSPHRR | Xenopus | BC074295 |
| | RSRSPRRHRSSSISPSRLKDRRDDDKKEPKESKGGGSKERQLAAEDLEG | laevis | |
| | KTEEEIEMMKLMGFASFDSSKGKKTDGSVNAYAINVSQKRKYRQYMNRK | (African | |
| | GGFNRPLDFVA | clawed | |
| | | frog) | |

In other configurations of the present teachings, the inventors disclose nucleic acid vectors comprising a promoter operably linked to a nucleic acid sequence encoding an oligopeptide comprise a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, and SEQ ID NO: 29. In some aspects, the sequence can be a sequence sharing at least about 70% sequence identity, at least about 75% sequence identity, at least about 80% sequence identity, at least about 85% sequence identity, and least about 90% sequence identity, at least about 95% sequence identity, at least about 96% sequence identity, at least about 97% sequence identity, at least about 98% sequence identity, or at least about 99% sequence identity with at least one of SEQ ID NO: 1 through SEQ ID NO: 29. The promoter can be a eukaryotic promoter (i.e., a promoter which can support transcription in the environment of a eukaryotic cell such as a mammalian cell or a microbial eukaryotic cell such as a yeast cell) or a prokaryotic promoter (i.e., a promoter which can support transcription in the environment of a prokaryotic cell such as a bacterium). Non-limiting examples of a promoter which can be used in a vector of the present teachings include an actin promoter, a CUP1 promoter from a yeast metallothionein gene, and promoter-enhancer elements from the simian virus 40 (SV40) early-region or a mouse alpha 2(l)-collagen gene, and an E. coli lac operon operator/promoter. A vector can be, for example, a plasmid or a virus, such as, for example, a baculovirus or a bacteriophage. In addition, in some configurations, the present teachings encompass a cell comprising a vector as described herein. A cell comprising a vector can be a cell in which the promoter of the vector is operable, for example an E. coli cell harboring a plasmid comprising a lac operon/promoter, or an insect cell harboring a baculovirus vector.

In various configurations, the present teachings include methods of treating (e.g., reducing or ameliorating) a bone disease, disorder, or injury in a subject in need thereof. Also, the present teachings include methods of promoting bone growth in a subject in need thereof. Methods of these configurations include administering, to a subject in need, a therapeutically effective amount of (i) a Mesd polypeptide (e.g., SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38); (ii) a polypeptide sharing at least about 70% sequence identity, at least about 75% sequence identity, at least about 80% sequence identity, at least about 85% sequence identity, and least about 90% sequence identity, at least about 95% sequence identity, at least about 96% sequence identity, at least about 97% sequence identity, at least about 98% sequence identity, or at least about 99% sequence identity with a Mesd polypeptide (e.g., SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38); (iii) an oligopeptide comprising between about 10 contiguous amino acids and about 70 contiguous amino acids, wherein the oligopeptide comprises an amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; and (iv) an oligopeptide comprising between 10 contiguous amino acids and about 70 contiguous amino acids, wherein the oligopeptide comprises an amino acid sequence sharing at least about 70% sequence identity, at least about 75% sequence identity, at least about 80% sequence identity, at least about 85% sequence identity, and least about 90% sequence identity, at least about 95% sequence identity, at least about 96% sequence identity, at least about 97% sequence identity, at least about 98% sequence identity, or at least about 99% sequence identity with at least one sequence set forth as SEQ ID NO: 1 through SEQ ID NO: 29. Generally, the polypeptide or oligopeptide employed in the methods of reducing or ameliorating bone disease or promoting bone growth in a subject in need thereof antagonizes binding of DKK1 to LRP5 when in contact with LRP5 and/or enhances Wnt signaling when such signaling is inhibited by an extracellular ligand such as DKK1, such as when a cell subjected to Wnt signaling is contacted with the oligopeptide.

The oligopeptide for use in therapeutic methods described above can be, for example, about 10 contiguous amino acids, about 15 contiguous amino acids, about 20 contiguous amino acids, about 25 contiguous amino acids, about 30 contiguous amino acids, about 35 contiguous amino acids, about 40 contiguous amino acids, about 45 contiguous amino acids, about 50 contiguous amino acids, about 55 contiguous amino acids, about 60 contiguous amino acids, or about 70 contiguous amino acids. For example, the oligopeptide can be up to 67 contiguous amino acids. Various embodiments of the oligonucleotide are discussed more fully above.

Bone disease, conditions, and injuries treatable by methods described herein include, but are not limited to, bone spurs, bone tumors, bone metastasis, craniosynostosis, enchondroma, fibrous dysplasia, McCune-Albright syndrome, giant cell tumor of bone, Klippel-Feil syndrome, scoliosis, osteitis condensans ilii, osteochondritis dissecans, osteogenesis imperfecta, otospondylomegaepiphyseal dysplasia, Weissenbacher-Zweymüller syndrome, Pallister-Hall syndrome, Greig cephalopolysyndactyly syndrome, McKusick-Kaufman syndrome, Bardet-Biedl syndrome, Oro-facial digital syndromes achondrogenesis, atelosteogenesis, Paget's disease, diastrophic dysplasia, recessive multiple epiphyseal dysplasia, spondyloperipheral dysplasia, ankylosing spondylitis, osteochondroma, osteomyelitis, osteopetroses, renal osteodystrophy, septic arthritis, unicameral bone cyst, osteomalacia, osteoporosis, osteoarthritis, total joint replacement, partial joint replacement, alveolar process-related tooth mobility, alveolar process-related tooth loss, periodontitis, and bone fracture. Treatment methodology described herein can accompany surgical intervention for the treatment of various bone disease, conditions, and injuries described above. Preferably, the bone disease treated by methods described herein is a degenerative bone disease, and more preferably, osteoporosis.

These methods can also be applied to cells or tissues in vitro or ex vivo, such as, in non-limiting example, osteoblasts grown under standard laboratory conditions.

In addition, in some aspects, the present methods also include administering to a subject in need of treatment a vector such as described above, or cells comprising a vector, such as human cells comprising a vector comprising a eukaryotic promoter operably linked to a nucleic acid encoding an oligopeptide as described herein. In non-limiting example, the human cells can be autologous osteoblasts from a subject which are transformed with a vector, grown in vitro using standard cell culture techniques, and returned to the donor.

A determination of the need for treatment will typically be assessed by a history and physical exam consistent with the bone disease or disorder at issue. Such diagnosis is within the skill of the art. Subjects with an identified need of therapy include those with a diagnosed bone disease or disorder or an indication of a bone disease or disorder amenable to therapeutic treatment described herein and subjects who have been treated, are being treated, or will be treated for a bone disease or disorder. For example, the diagnosis of a degenerative bone disease, such as osteoporosis, can serve to identify a subject with a need for a therapy described herein. The subject is preferably an animal, including, but not limited to, mammals, reptiles, and avians, more preferably horses, cows, dogs, cats, sheep, pigs, and chickens, and most preferably human.

The polypeptides or oligonucleotides of the invention, as discussed above, can also be used in the manufacture of a medicament for the treatment of a bone disease, disorder, or injury. Similarly, the polypeptides or oligonucleotides of the invention can be used in the manufacture of a medicament for promotion of bone growth.

A polypeptide, oligopeptide, or vector described herein can be formulated by any conventional manner using one or more pharmaceutically acceptable carriers and/or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of the polypeptides, oligopeptides, or vectors, preferably in a substantially purified form, together with a suitable amount of carrier so as to provide the form for proper administration to the subject. The formulation should suit the mode of administration. The agents of use with the current invention can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents of the present invention and/or together with other biologically active or biologically inert agents. Such biologically active or inert agents may be in fluid or mechanical communication with the agent(s) or attached to the polypeptides, oligopeptides, or vectors by ionic, covalent, Van der Waals, hydrophobic, hydrophillic or other physical forces.

When used in the treatments described herein, a therapeutically effective amount of a polypeptide, oligopeptide, or vector of the present invention may be employed in a substantially pure form or, where such forms exist, in pharmaceutically acceptable salt form and with or without a pharmaceutically acceptable excipient. For example, the polypeptide, oligopeptide, or vector of the invention can be administered, at a reasonable benefit/risk ratio applicable to any medical treatment, in the stimulation of bone formation in the subject by, for example, antagonization of binding of DKK1 to LRP5 when in contact with LRP5.

Toxicity and therapeutic efficacy of such agents can be determined by standard pharmaceutical procedures in cell cultures and/or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where large therapeutic indices are preferred.

A therapeutically effective amount of a polypeptide, oligopeptide or vector of the present teachings can be determined using methods well known in the art, such as found in standard pharmaceutical texts such as Herfindal, Gourley and Hart, Williams and Wilkins, ed. Clinical Pharmacy and Therapeutics, Williams & Wilkins, 1988; Goodman, L. S. and Gilman, A., ed. The Pharmacological Basis of Therapeutics, McGraw-Hall;, 2005; Kalant, H., and Roschlau, W. H. E., ed., Principles of Medical Pharmacology, Mosby, Incorporated. 1989; J. T. DiPiro, R. L. et al., ed. Pharmacotherapy: A Pathophysiologic Approach, McGraw-Hill Medical Publishing, 2005; Ascione, Principles of Scientific Literature Evaluation Critiquing Clinical Drug Trials, American Pharmacists Association, 2001; and Remington, The Science and Practice of Pharmacy, Lippincott Williams & Wilkins, 2005.

The specific therapeutically effective dose level for any particular subject will depend upon a variety of factors including the disorder being treated and the severity of the disorder; activity of the specific polypeptide, oligopeptide, or vector employed; the specific composition employed; the age, body weight, general health, sex and diet of the subject; the time of administration; the route of administration; the rate of excretion of the specific polypeptide, oligopeptide, or vector employed; the duration of the treatment; drugs used in combination or coincidental with the specific polypeptide, oligopeptide, or vector employed and like factors well known in the medical arts. For example, it is well within the skill of the art to start doses of a polypeptide, oligopeptide, or vector at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. Specific dosages for each type of inhibitor are discussed more fully above. It will be understood, however, that the total daily usage of the polypeptide, oligopeptide, or vector s and compositions of the present invention will be decided by the attending physician within the scope of sound medical judgment.

The amount of a polypeptide, oligopeptide, or vector of the present invention that may be combined with a pharmaceutically acceptable carrier to produce a single dosage form will vary depending upon the subject treated and the particular mode of administration. It will be appreciated by those skilled in the art that the unit content of polypeptide, oligopeptide, or vector contained in an individual dose of each dosage form need not in itself constitute a therapeutically effective amount, as the necessary therapeutically effective amount could be reached by administration of a number of individual doses.

Administration of a polypeptide, oligopeptide, or vector of the invention can occur as a single event or over a time course of treatment. For example, modulators can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

A polypeptide, oligopeptide, or vector of the present invention can also be used in combination with other therapeutic modalities. Thus, in addition to the therapies described herein, one may also provide to the subject other therapies known to be efficacious for particular bone diseases, such as osteoporosis.

Controlled-release (or sustained-release) preparations may be formulated to extend the activity of a polypeptide, oligopeptide, or vector of the present invention and reduce dosage frequency. Controlled-release preparations can also be used to effect the time of onset of action or other characteristics, such as blood levels, and consequently affect the occurrence of side effects.

Controlled-release preparations may be designed to initially release an amount of a polypeptide, oligopeptide, or vector of the present invention that produces the desired therapeutic effect, and gradually and continually release other amounts to maintain the level of therapeutic effect over an extended period of time. In order to maintain a near-constant level in the body, the polypeptide, oligopeptide, or vector of the present invention can be released from the dosage form at a rate that will replace the amount being metabolized and/or excreted from the body. The controlled-release may be stimulated by various inducers, e.g., change in pH, change in temperature, enzymes, water, or other physiological conditions or molecules.

Controlled-release systems may include, for example, an infusion pump which may be used to administer a polypeptide, oligopeptide, or vector of the present invention in a manner similar to that used for delivering insulin or chemotherapy to specific organs or tumors. Typically, using such a system, a polypeptide, oligopeptide, or vector of the present invention is administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

A polypeptide, oligopeptide, or vector for use in the methods described herein can be delivered in a variety of means known to the art. A polypeptide, oligopeptide, or vector of the invention can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

A polypeptide, oligopeptide, or vector of the present invention may be administered by controlled-release means or delivery devices that are well known to those of ordinary skill in the art. These include, for example, hydropropylmethyl cellulose, other polymer matrices, gels, permeable membranes, osmotic systems, multilayer coatings, microparticles, liposomes, microspheres, or the like, or a combination of any of the above to provide the desired release profile in varying proportions. Other methods of controlled-release delivery of agents will be known to the skilled artisan and are within the scope of the invention.

A polypeptide, oligopeptide, or vector of the present invention can be administered through a variety of routes well known in the arts. Examples include methods involving direct injection (e.g., systemic or stereotactic), implantation of cells engineered to secrete the factor of interest, drug-releasing biomaterials, implantable matrix devices, implantable pumps, injectable gels and hydrogels, liposomes, micelles (e.g., up to 30 μm), nanospheres (e.g., less than 1 μm), microspheres (e.g., 1-100 μm), reservoir devices, etc.

A polypeptide, oligopeptide, or vector of the present invention can be encapsulated and administered in a variety of carrier delivery systems. Examples of carrier delivery systems include microspheres, hydrogels, polymeric implants, smart ploymeric carriers, and liposomes. Carrier-based systems for polypeptide, oligopeptide, or vector delivery can: provide for intracellular delivery; tailor release rates; increase the proportion of biomolecule that reaches its site of action; improve the transport of the drug to its site of action; allow colocalized deposition with other agents or excipients; improve the stability of the biomolecule in vivo; prolong the residence time of the biomolecule at its site of action by reducing clearance; decrease the nonspecific delivery of the biomolecule to nontarget tissues; decrease irritation caused by the biomolecule; decrease toxicity due to high initial doses of the biomolecule; alter the immunogenicity of the biomolecule; decrease dosage frequency, improve taste of the product; and/or improve shelf life of the product.

Polymeric microspheres can be produced using naturally occurring or synthetic polymers and are particulate systems in the size range of 0.1 to 500 pm. Polymeric micelles and polymeromes are polymeric delivery vehicles with similar characteristics to microspheres and can also facilitate encapsulation and delivery of a polypeptide, oligopeptide, or vector described herein. Fabrication, encapsulation, and stabilization of microspheres for a variety of polypeptide, oligopeptide, or vector payloads are within the skill of the art (see e.g., Varde & Pack (2004) Expert Opin. Biol. 4(1) 35-51). Release rate of microspheres can be tailored by type of polymer, polymer molecular weight, copolymer composition, excipients added to the microsphere formulation, and microsphere size. Polymer materials useful for forming microspheres include PLA, PLGA, PLGA coated with DPPC, DPPC, DSPC, EVAc, gelatin, albumin, chitosan, dextran, DL-PLG, SDLMs, PEG (e.g., ProMaxx), sodium hyaluronate, diketopiperazine derivatives (e.g., Technosphere), calcium phosphate-PEG particles, and oligosaccharide derivative DPPG (e.g., Solidose). Encapsulation can be accomplished, for example, using a water/oil single emulsion method, a water-oil-water double emulsion method, or lyophilization. Several commercial encapsulation technologies are available (e.g., ProLease®, Alkerme). Microspheres encapsulating a polypeptide, oligopeptide, or vector of the present invention can be administered in a variety of means including parenteral, oral, pulmonary, implantation, and pumping device.

Polymeric hydrogels, composed of hydrophillic polymers such as collagen, fibrin, and alginate, can also be used for the sustained release of a polypeptide, oligopeptide, or vector of the present invention (see generally, Sakiyama et al. (2001) FASEB J. 15,1300-1302).

Three-dimensional polymeric implants, on the millimeter to centimeter scale, can be loaded with a polypeptide, oligopeptide, or vector of the present invention (see generally, Teng et al (2002) Proc. Natl. Acad. Sci. U.S.A. 99, 3024-3029). A polymeric implant typically provides a larger depot of the bioactive factor. The implants can also be fabricated into structural supports, tailoring the geometry (e.g., shape, size, porosity) to the application. Implantable matrix-based delivery systems are also commercially available in a variety of sizes and delivery profiles (e.g., Innovative Research of America, Sarasota, Fla.).

“Smart” polymeric carriers can be used to administer polypeptide, oligopeptide, or vector of the present invention (see generally, Stayton et al. (2005) Orthod Craniofacial Res 8, 219-225; Wu et al. (2005) Nature Biotech (2005) 23(9), 1137-1146). Carriers of this type utilize polymers that are hydrophilic and stealth-like at physiological pH, but become hydrophobic and membrane-destabilizing after uptake into the endosomal compartment (i.e., acidic stimuli from endosomal pH gradient) where they enhance the release of the cargo molecule into the cytoplasm. Design of the smart polymeric carrier can incorporate pH-sensing functionalities, hydrophobic membrane-destabilizing groups, versatile conjugation and/or complexation elements to allow the drug incorporation, and an optional cell targeting component. Potential therapeutic macromolecular cargo includes a polypeptide, oligopeptide, or vector of the present invention. As an example, smart polymeric carriers, internalized through receptor mediated endocytosis, can enhance the cytoplasmic delivery of a polypeptide, oligopeptide, or vector of the present invention, and/or other agents described herein. Polymeric carriers include, for example, the family of poly(alkylacrylic acid) polymers, specific examples including poly(methylacrylic acid), poly(ethylacrylic acid) (PEAA), poly(propylacrylic acid) (PPAA), and poly(butylacrylic acid) (PBAA), where the alkyl group progressively increased by one methylene group. Smart polymeric carriers with potent pH-responsive, membrane destabilizing activity can be designed to be below the renal excretion size limit. For example, poly(EAA-co-BA-co-PDSA) and poly(PAA-co-BA-co-PDSA) polymers exhibit high hemolytic/membrane destabilizing activity at the low molecular weights of 9 and 12 kDa, respectively. Various linker chemistries are available to provide degradable conjugation sites for proteins, nucleic acids, and/or targeting moieties. For example, pyridyl disulfide acrylate (PDSA) monomer allow efficient conjugation reactions through disulfide linkages that can be reduced in the cytoplasm after endosomal translocation of the therapeutics.

Liposomes can be used to administer agents that decrease levels of Ahal and/or other related molecules with similar function. The drug carrying capacity and release rate of liposomes can depend on the lipid composition, size, charge, drug/lipid ratio, and method of delivery. Conventional liposomes are composed of neutral or anionic lipids (natural or synthetic). Commonly used lipids are lecithins such as (phosphatidylcholines), phosphatidylethanolamines (PE), sphingomyelins, phosphatidylserines, phosphatidylglycerols (PG), and phosphatidylinositols (PI). Liposome encapsulation methods are commonly known in the arts (Galovic et al. (2002) Eur. J. Pharm. Sci. 15, 441-448; Wagner et al. (2002) J. Liposome Res. 12, 259-270). Targeted liposomes and reactive liposomes can also be used to deliver the biomolecules of the invention. Targeted liposomes have targeting ligands, such as monoclonal antibodies or lectins, attached to their surface, allowing interaction with specific receptors and/or cell types. Reactive or polymorphic liposomes include a wide range of liposomes, the common property of which is their tendency to change their phase and structure upon a particular interaction (eg, pH-sensitive liposomes) (see e.g., Lasic (1997) Liposomes in Gene Delivery, CRC Press, Fla.).

Various other delivery systems are known in the art and can be used to administer the agents of the invention. Moreover, these and other delivery systems may be combined and/or modified to optimize the administration of the agents of the present invention.

The methods and compositions having been described herein utilize laboratory techniques well known to skilled artisans and can be found in references such as Sambrook and Russel (2006), Condensed Protocols from Molecular Cloning: A Laboratory Manual, Cold Spring Harbor Laboratory Press, ISBN 0879697717; Sambrook and Russel (2001) Molecular Cloning: A Laboratory Manual, Cold Spring Harbor Laboratory Press, ISBN 0879695773; Ausubel et al. (2002) Short Protocols in Molecular Biology, Current Protocols, ISBN 0471250929; Spector et al. (1998) Cells: A Laboratory Manual, Cold Spring Harbor Laboratory Press, ISBN 0879695226. For pharmaceutical compositions and methods of treatment disclosed herein, dosage forms and administration regimes can be determined using standard methods known to skilled artisans, for example as set forth in standard references such as Remington's Pharmaceutical Sciences, 21st edition (A. R. Gennaro, Ed.) (2005) Lippincott Williams & Wilkins, ISBN: 0781746736; Hardman, J. G., et al., Goodman & Gilman's The Pharmacological Basis of Therapeutics, Ninth Edition, McGraw-Hill, 1996; and Rowe, R. C., et al., Handbook of Pharmaceutical Excipients, Fourth Edition, Pharmaceutical Press, 2003.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### REFERENCES CITED

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Mesd Binds to Mature LRP6 at the Cell Surface

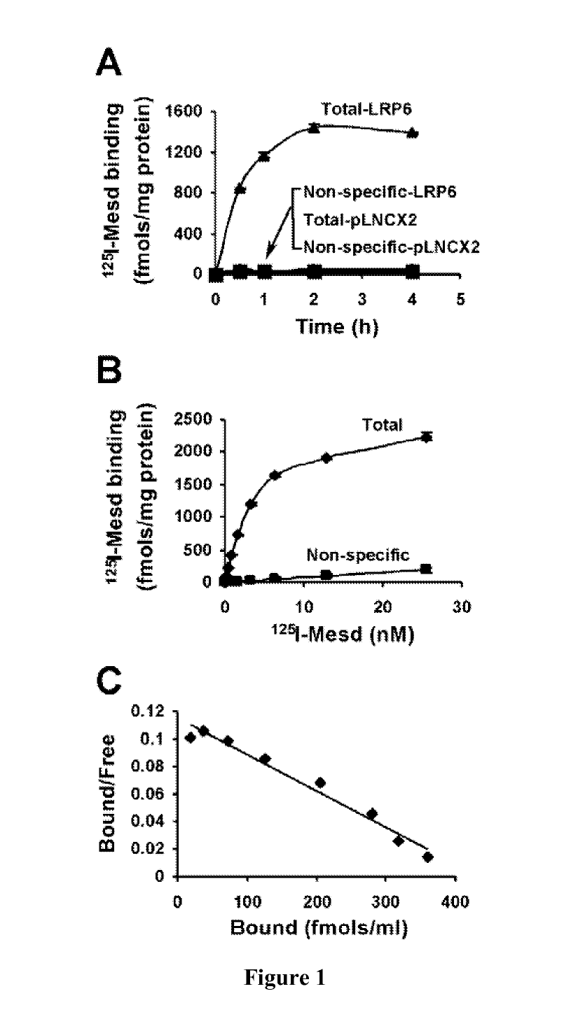

To examine whether Mesd binds with high affinity to most members of the LDLR family at the cell surface, cell surface ligand binding experiments were performed with cells stably transduced with LRP6 cDNA. Human HT1080 cells, which express undetectable levels of LRP6, were transduced with a viral vector alone (pLNCX2) or with vector containing LRP6 cDNA (Li, Y., Oncogene 23: 9129-9135, 2004) and used for ¹²⁵I-Mesd binding (FIG. 1A). ¹²⁵I-Mesd (5 nM) reached maximal binding after 2 hours incubation at 4° C. with LRP6-expressing HT1080 cells (FIG. 1A). Inclusion of excess unlabeled Mesd (500 nM) completely eliminated this binding. No significant ¹²⁵I-Mesd binding was seen with the control cells (pLNCX2). Saturation of Mesd specific binding was seen at concentrations of >6.4 nM (FIG. 1 B). Scatchard analysis of the binding data revealed that Mesd binds LRP6 with a Kd of ˜3.3 nM (FIG. 1C). This affinity of Mesd to LRP6 is comparable to that of RAP to LRP (ladonato, S. P., Biochem. J. 296: 867-875,1993).

As illustrated in FIG. 1, Mesd binds to mature LRP6 at the cell surface with high affinity. (A) Time course of ¹²⁵I-Mesd (5 nM) binding to LRP6-transduced HT1080 cells and the control cells. Assay was carried out for the indicated periods at 4° C. in the absence (total) or presence of 500 nM Mesd (non-specific). (B) Saturation binding of ¹²⁵I-Mesd to LRP6-transduced HT1080 cells and the control cells. Assay was carried out at indicated concentrations for 3 hours at 4° C. in the absence (total) or presence (non-specific) of 500 nM Mesd. (C) Scatchard plots of data in B. All values are the average of triple determinations with the s.d. indicated by error bars.

In this and all subsequent examples, the following materials and methods were used:

### Materials

Human recombinant DKK1 protein and mouse recombinant Wnt3a protein were from R&D Systems. Human recombinant RAP protein was expressed in a glutathione S-transferase (GST) expression vector and isolated as described previously (Bu et al., 1993). Monoclonal anti-Myc antibody 9E10 was from Roche. Monoclonal antibody 8G1 against human LRP was from Research Diagnostics. Monoclonal anti-HA antibody has been described before (Li, Y., J. Biol. Chem. 275: 17187-17194, 2000). Polyclonal rabbit anti-LDLR was produced by immunizing rabbits with recombinant human LDLR1-294 fragment. Peroxidase-labeled antimouse antibody and ECL system were from Amersham Life Science. Plasmid pcDNA3.1C-Myc-hLRP5 containing the full-length human LRP5 cDNA and plasmid pCS-Myc-hLRP6 containing the full-length human LRP6 cDNA were from Cindy Bartels and Christof Niehrs, respectively. Carrier-free Na¹²⁵I was purchased from NEN Life Science Products. IODO-GEN was from Pierce. Proteins were iodinated by using the IODO-GEN method as described previously (Li, Y., J. Biol. Chem. 275: 17187-17194, 2000).

### Cell Lines and Cell Culture

LRP6-transduced HT1080 cells and the control cells have been described before (Li et al., 2004), and were cultured in DMEM medium containing 10% fetal bovine serum and 350_g/ml G418. The LRP-null CHO cells stably transfected with human LDLR-related protein (LRP) minireceptor mLRP4, mLRP4 tail mutant mLRP4tailess (mLRP4 without the cytoplasmic tail), human LDLR-related protein 1 B (LRP1 B) minireceptor mLRP1 B4, human VLDLR, or human apoER2 have been described before (Li, Y., J. Biol. Chem. 275: 17187-17194, 2000; Li et al., 2001; Liu et al., 2001), and were cultured in Ham's F-12 medium containing 10% fetal bovine serum and 350 μg/ml G418. A set of genetically derived murine embryonic fibroblasts (MEF) from mouse embryos deficient for LRP and/or LDLR were obtained from Joachim Herz, University of Texas Southwestern Medical Center at Dallas (Willnow, J. Cell Sci. 107: 719-726., 1994; Narita, M., J. Biochem. 132: 743-749, 2002). These are MEF-1 (WT), MEF-2 (LRP-deficient), MEF-3 (LDLR-deficient), and MEF-4 (LRP and LDLR-double-deficient), and are cultured in DMEM containing 10% fetal bovine serum. Culture conditions of U87, MCF-7, and human aortic smooth muscle cells have been described before (Li, Y., FEBS Lett. 555: 346-350, 2003). HEK293 cells were from ATCC, and cultured in DMEM containing 10% fetal bovine serum.

### Preparation of Recombinant Mesd Protein

Full-length mouse Mesd cDNA was obtained. The wild-type and mutant forms of mouse Mesd were generated by polymerase chain reactions, and subcloned into the expression vector pET-30a(+) (Novagen) at the EcoRi and HindIII restriction sites. The integrity of the subcloned DNA sequence was confirmed by DNA sequencing. Recombinant proteins were overexpressed from pET-30(+)Mesd in E. coli. BL21 (DE3) producing a recombinant fusion protein with a polyhistidine metal-binding tail at the N-terminus, and purified with His-Bind Kits from Novagen according to the manufacturer's protocol. All the recombinant Mesd proteins lack the Mesd signal peptide.

### Western Blotting

To examine the expression of the LDLR family members, cells cultured in six-well plates were lysed with 0.5 ml lysis buffer (phosphate-buffered saline containing 1% Triton X-100 and 1 mM PMSF) at 4° C. for 30 minutes. Equal quantities of protein were subjected to SDS-PAGE under non-reducing conditions. Following transfer to Immobilon-P membrane, successive incubations with primary antibody and horseradish peroxidase-conjugated secondary antibody were carried out for 60 minutes at room temperature. The immunoreactive proteins were then detected using the ECL system.

To examine the cytosolic-catenin level, cells in six-well plates were treated with Mesd at various concentrations for 90 minutes at 37° C. After washing in ice-cold PBS, cells were collected and homogenized in a glass Dounce homogenizer in buffer consisting of 100 mM Tris-HCl pH 7.4, 140 mM NaCl, 2 mM DTT, 2 mM PMSF, and 1X Complete™ protease inhibitors (500 μl/well). The homogenate was centrifuged for 10 minutes at 500 g, and the supernatant was further centrifuged at 100,000 g at 4° C. for 90 minutes. The resulting supernatant was designated the cytosolic fraction. The β-catenin levels were then examined by western blotting using β-catenin-specific antibody from Cell Signaling Technology. The immunoreactive proteins were detected using the ECL system. Films showing immunoreactive bands were scanned with a Kodak Digital Science DC120 Zoom Digital Camera and band intensities were analyzed with Kodak Digital Science 1D Image Analysis Software.

### Luciferase Reporter Assay

HEK293 cells were plated into six-well plates. For each well, 0.1 pg of the TOP-FLASHTCF luciferase construct (Upstate Biotechnology) was cotransfected with 0.8 μg Mesd-expressing vector, 0.8 μg Mesd mutant-expressing vector, or empty vector. A β-galactosidase-expressing vector (Promega, Madison, Wis.) was included as an internal control for transfection efficiency. After 48 hours, cells were lysed and both luciferase and β-galactosidase activities were determined with enzyme assay kits (Promega). The luciferase activity was determined with a luminometer using the Dual Luciferase Assay system (Promega). Luciferase activity was normalized to the activity of the P-galactosidase.

### Ligand Binding and Degradation

Cells (2×10⁵) were seeded into 12-well dishes 1 day prior to assay. Ligand-binding buffer (minimal Eagle's medium containing 0.6% BSA with a different concentration of radioligand, 0.6 ml/well) was added to cell monolayers, in the absence or the presence of 500 nM unlabeled RAP or 500 nM unlabeled Mesd, followed with incubation for 0-4 hours at 4° C. Thereafter, overlying buffer containing unbound ligand was removed, and cell monolayers were washed and lysed in low-SDS lysis buffer (62.5 mM Tris-HCl pH 6.8, 0.2% SDS, 10% v/glycerol) and counted. The protein concentration of each cell lysate was measured in parallel dishes that did not contain the ligands.

Ligand degradation was performed using the methods as described (Li, Y., J. Biol. Chem. 275: 17187-17194, 2000). Briefly, 2×10⁵ cells were seeded into 12-well dishes 1 day prior to assay. Pre-warmed assay buffer (minimal Eagle's medium containing 0.6% BSA with radioligand, 0.6 ml/well) was added to cell monolayers in the absence or the presence of unlabeled 500 nM RAP or 500 nM Mesd, followed by incubation for 4 hours at 37° C. Thereafter, the medium overlying the cell monolayers was removed and proteins were precipitated by addition of BSA to 10 mg/ml and trichloroacetic acid to 20%. Degradation of radioligand was defined as the appearance of radioactive fragments in the overlying medium that were soluble in 20% trichloroacetic acid. Kinetic analysis of endocytosis LRP6-transduced HT1080 cells were plated in 12-well plates at a density of 2×10⁵ cells/well and used after overnight culture. Cells were rinsed twice in ice-cold assay buffer (minimal Eagle's medium containing 0.6% BSA), and ¹²⁵I-anti-HA IgG was added at 1 nM final concentration in cold assay buffer (0.5 ml/well). The binding of ¹²⁵Ianti- HA IgG was carried out at 4° C. for 90 minutes with gentle rocking. Unbound ¹²⁵I-anti-HA IgG was removed by washing cell monolayers three times with cold assay buffer. Ice-cold stop/strip solution (0.2 M acetic acid, pH 2.6, 0.1 M NaCl) was added to one set of plates without warming up and kept on ice. The remaining plates were then placed in a 37° C. water bath and 0.5 ml assay buffer prewarmed to 37° C. was quickly added to cell monolayers to initiate internalization. After each time point, the plates were quickly placed on ice and the assay buffer was replaced with cold stop/strip solution. ¹²⁵I-anti-HA IgG that remained on the cell surface was stripped by incubation of cell monolayers with cold stop/strip solution for a total of 20 minutes (0.75 ml for 10 minutes, twice) and counted. Cell monolayers were then solubilized with low-SDS lysis buffer and counted. The sum of ¹²⁵I-anti-HA IgG that was internalized plus that remaining on the cell surface after each assay was used as the maximum potential internalization. The fraction of internalized ¹²⁵I-anti-HA IgG after each time point was calculated and plotted.

### Cell Surface DKK1 Binding and Immunodetection

Human DKK1 cDNA (clone MGC:868, IMAGE:3508222) was obtained from Invitrogen and subcloned into pcDNA3 (EcoRI/Xbal). To facilitate immunodetection, a c-Myc epitope was included at the C-terminus. The integrity of the subcloned DNA sequence was confirmed by DNA sequencing. Human DKK1-conditioned media were produced by transient transfection of HEK293 cells with pcDNADKK1-Myc in serum-free medium, and allowed to bind to LRP6-transduced HT1080 cells and control cells at room temperature for 60 minutes in the absence or presence of 1 μM Mesd. Cells were then fixed in 4% paraformaldehyde, labeled with anti-Myc monoclonal antibody and detected with Alexa-488 goat anti-mouse IgG. The immunofluorescence was detected by a laser-scanning confocal microscope (Olympus Fluoview 500).

### Example 2

### MESD Binds to Matur LRP5/6 but not Significantly to Other Members of the LDLR Family

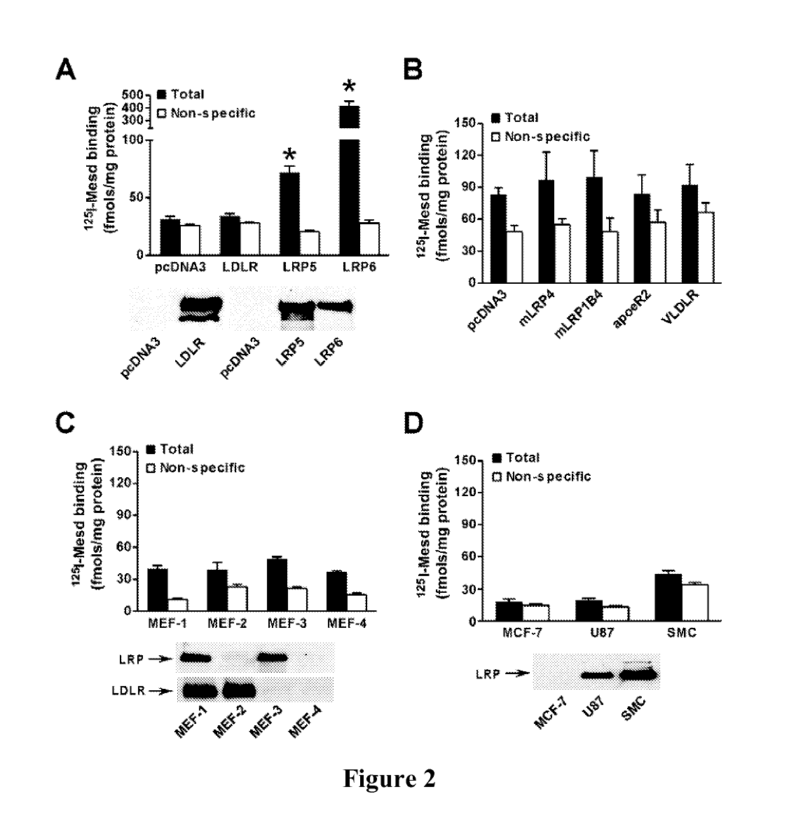

To determine whether Mesd binds to other members of the LDLR family, ¹²⁵I-Mesd binding analysis was performed with four groups of cells expressing different members of the LDLR family (FIG. 2). In the first experiment, HEK293 cells were transiently transfected with cDNAs for the LDLR, LRP5, LRP6 or empty pcDNA3 vector. In the second experiment, LRP-null Chinese hamster ovary (CHO) cells were stably transfected with LRP minireceptor mLRP4, LRP1 B minireceptor mLRP1 B4, apoER2, VLDLR, or empty pcDNA3 vector (Li, Y., J. Biol. Chem. 275: 17187-17194, 2000; Li, Y., J. Biol. Chem. 276: 18000-18006, 2001; Liu, C. X., J. Biol. Chem. 276:28889-28896, 2001). mLRP4 is composed of residues 3274-4525 of the full-length LRP, which includes the fourth cluster of ligand-binding repeats and the entire C-terminus of the receptor. mLRP1 B4 is composed of residues 3276-4599 of the full length LRP1 B, which includes the fourth cluster of ligand binding repeats and the entire C-terminus of the receptor mLRP4 and mLRP1 B4 mimic the function and trafficking of LRP and LRP1 B, respectively. In the third experiment, wild-type murine embryonic fibroblasts and murine embryonic fibroblasts with genetic deficiency of LDLR, LRP, or both (Willnow, T. E., J. Cell Sci. 107: 719-726, 1994; Narita, J. Biochem. 132: 743-749, 2002) were used. In the fourth experiment, the human breast cancer cell line MCF-7, human glioblastoma cell line U87, and human aortic smooth muscle cells (SMC) were used. MCF-7 cells express LRP at an undetectable level, whereas U87 cells and SMC express abundant LRP (Li, Y., FEBS Lett. 555: 346-350, 2003). Interestingly, among the members of the LDLR family examined, only LRP5 specifically binds to Mesd, albeit at lower levels compared to LRP6 when these receptors were expressed at comparable levels (FIG. 2A). Although there is a suggestion of Mesd binding to LRP when examined in CHO and MEF cells (FIG. 2B,C), specific Mesd binding to U87 or SMC, both of which express abundant LRP, was minimal (FIG. 2D). Therefore, specific binding of Mesd to CHO and MEF cells may reflect endogenous LRP5/LRP6 in these cells.

As illustrated in FIG. 2, Mesd binds to mature LRP5/6 but not significantly to other members of the LDLR family. (A) Binding of ¹²⁵I-Mesd (5 nM) to HEK293 cells transient transfected with human HA-tagged LDLR, Myc-tagged LRP5, Myc-tagged LRP6 or control vector. Lower panel, western blot analysis for the expression of the LDLR, LRP5 and LRP6. Equal amounts of cell lysate were applied for each lane. (B) Binding of ¹²⁵I-Mesd (5 nM) to LRP-null CHO cells stably transfected with LRP minireceptor mLRP4, LRP1 B minireceptor mLRP1 B4, VLDLR, apoER2 or empty pcDNA3 vector only. (C) Binding of ¹²⁵I-Mesd (5 nM) to wild-type murine embryonic fibroblasts (MEF-1) or MEF cell lines genetically deficient in LRP (MEF-2), LDLR (MEF-3) or both (MEF-4). Lower panel, western blot analysis of LRP and the LDLR expression in MEF cell lines. (D) Binding of ¹²⁵I-Mesd (5 nM) to human breast cancer cell line MCF-7, human glioblastoma cell line U87 and human aortic smooth muscle cells (SMC). Lower panel, western blot analysis of LRP expression in these cell lines. Assays were carried out for 4 hours at 4° C. in the absence (total) or presence of 500 nM Mesd. Values are the means of triple determinations with the s.d. indicated by error bars.

### Example 3

### The Carboxy-Terminal Region of MESD is Required for LRP6 Folding

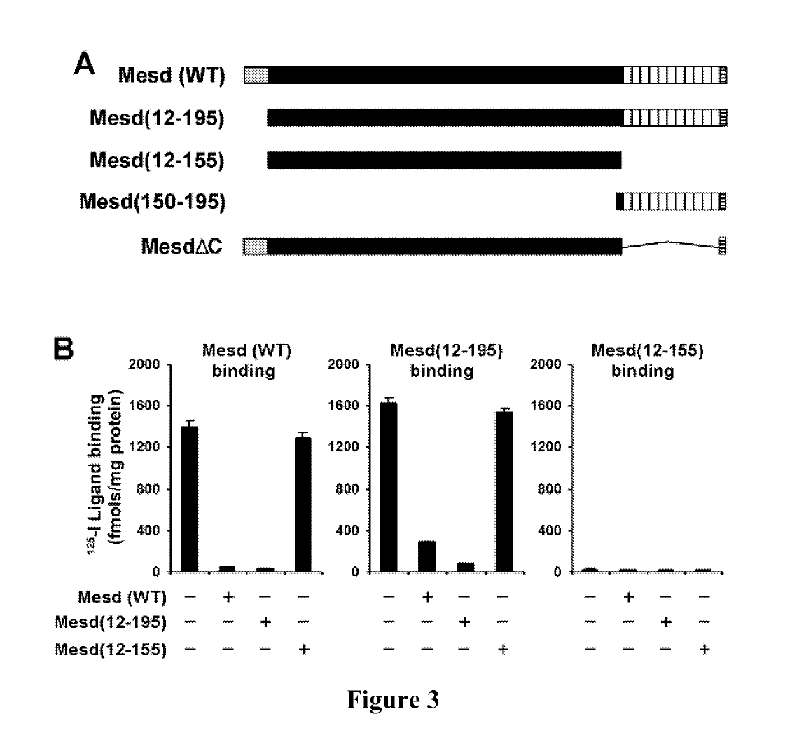

To analyze the Mesd sequences that are required for Mesd to bind to mature LRP6 at the cell surface with high affinity, sequences were compared between mouse Mesd and its homologs from different species. It was found that the first 12 amino acids of mouse Mesd are absent in the nematode worms Caenorhabditis elegans and Caenorhabditis briggsae, and that mouse Mesd, as well as human Mesd, has an extra ˜30 amino acid fragment prior to the conserved endoplasmic reticulum retention signal in its C-terminus (Culi, J., Cell 112: 343-354, 2003; Hsieh, J. C., Cell 112: 355-367, 2003). We thus generated two truncated Mesd mutants lacking either the N-terminal region, MESD(12-195), or both the N-terminal and C-terminal regions, Mesd(12-155) (FIG. 3A). The ability of these mutants to bind to cell surface LRP6 was then assessed. It was found that although truncation of the N-terminal 11 amino acids of mouse Mesd had no effect on LRP6 binding, further truncation of the last 40 amino acids completely abolished LRP6 binding (FIG. 3B).

### Example 4

### 45 Amino Acids of MESD are Necessary and Sufficient for Binding to Mature LRP6

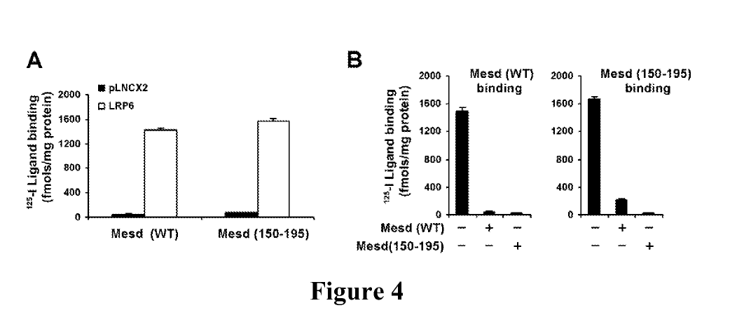

In this example, a truncated Mesd mutant containing the last 45 amino acids of C-terminal region was generated (FIG. 3A) and its binding affinity for LRP6 was then analyzed. As shown in FIG. 4, The C-terminal region of Mesd is necessary and sufficient for LRP6 binding. (A) Binding analyses of ¹²⁵I-Mesd and its mutant Mesd (150-195) (5 nM) to LRP6-transduced HT1080 cells and control cells. (B) Binding analyses of ¹²⁵I-Mesd and its mutant Mesd (150-195) (5 nM) to LRP6-transduced HT1080 cells. Assays were carried out for 3 hours at 4° C. in the absence or presence of 500 nM Mesd or its mutant. Values are the means of triple determinations with the s.d. indicated by error bars.

### Example 5

### Carboxy-Terminal Region of MESD is Required for LRP6 Folding

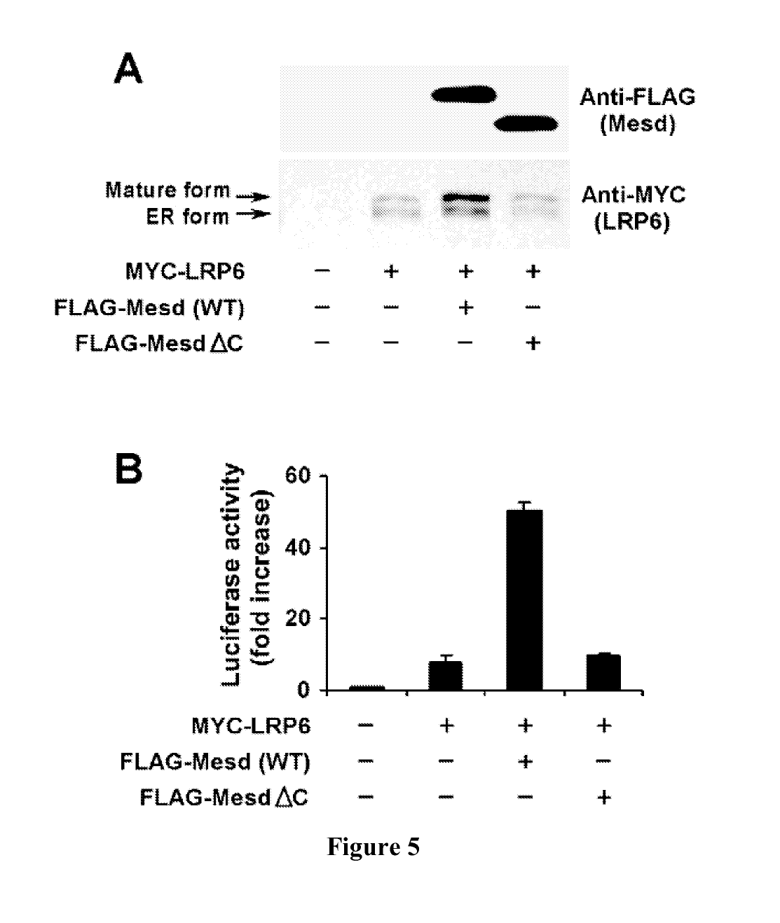

To examine the role of this C-terminal region of Mesd on receptor folding, a Mesd mutant (Mesd_C), which lacks the C-terminal region (amino acids 156-191) but retains the endoplasmic reticulum retention signal (REDL) was generated (FIG. 3A). Next, a potential role for MesdΔC on LRP6 folding was evaluated. HEK293 cells were transiently transfected with cDNA for the LRP6 with cotransfection of control vector, or cDNAs for Mesd or MesdΔC. The steady-state levels of LRP6 were analyzed by western blotting with the anti-MYC antibody (FIG. 5A). As seen in the figure, two forms of the receptor, i.e. the ER form and mature form (containing complex sugar modifications), were seen for LRP6. In the presence of Mesd coexpression, but not of Mesd_C coexpression, the amount of the mature form of LRP6 was significantly increased (FIG. 5A). In FIG. 5A, HEK293 cells were transiently transfected with the indicated cDNAs. Cell lysates were analyzed by SDS-PAGE under reducing conditions and western blotted with anti-FLAG or anti-HA antibodies as indicated.

Activation of canonical Wnt signaling leads to the stabilization of P-catenin and regulation of gene transcription through transcription regulators including lymphoid-enhancing factor (LEF)-1 and T-cell factors (TCF). The TOP-FLASH luciferase reporter contains TCF-binding sites and can be directly activated by the P-catenin/TCF complex (Korinek et al., 1997). LRP6 is cell surface receptor, and only the mature receptor can reach the cell surface and modulate Wnt signaling (Cong et al., 2004). Next examined was the effect of Mesd_C on Wnt signaling using the TOP-FLASH luciferase reporter assay in HEK293 cells. As expected, Mesd coexpression, but not MesdΔC coexpression, significantly enhanced TCF/LEF transcriptional activity (FIG. 5B). In FIG. 5B, HEK293 cells were cotransfected with LRP6, MESD, MesdΔC or empty pcDNA3 vector and a TCF/LEF transcriptional activity reporter plasmid (TOP-FLASH). The luciferase activity was measured 48 hours after transfection. Values are the means of triple determinations with the s.d. indicated by error bars. Together, these results suggest that the C-terminal region of Mesd is required for LRP6 folding and its signaling function at the cell surface.

### Example 6

### LRP6 is not a Constitutively Active Endocytosis Receptor and Mediates a Limited Level of MESD Degradation

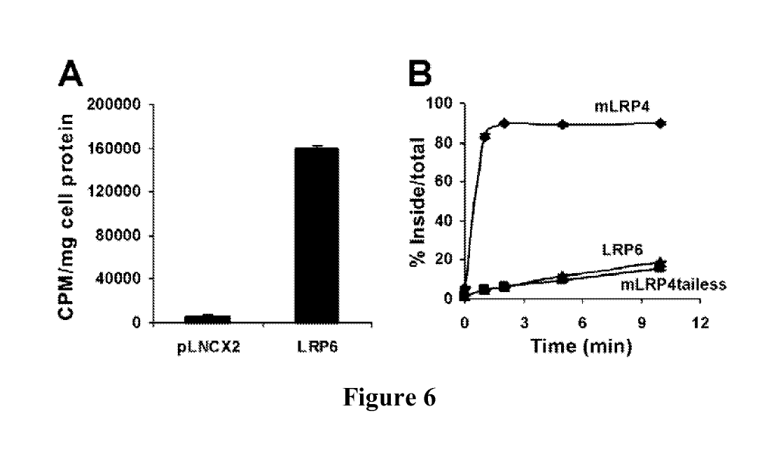

Cell surface receptors that traffic between the plasma membrane and endocytic compartments contain signals within their cytoplasmic tails that allow for efficient recruitment into endocytic vesicles. In many cases (e.g. LRP and the LDLR), these signals are constitutively active and mediate continuous receptor endocytosis independently of ligand binding. To examine whether LRP6 is a constitutively active endocytosis receptor, kinetic analyses of receptor endocytosis with HT1080 cells transduced with HA-tagged LRP6 were performed. To eliminate potential effects of LRP6 ligands on its internalization, we utilized ¹²⁵I-anti-HA IgG for LRP6 endocytosis assays. Binding of ¹²⁵I-anti-HA IgG to HA-tagged LRP6 was specific, i.e. the binding of ¹²⁵I-anti-HA IgG to the HT1080 control cells was minimal when compared to HT1080-LRP6 cells (FIG. 6A). We used HA-tagged LRP minireceptor mLRP4 as a positive control and mLRP4tailess (mLRP4 lacking the cytoplasmic tail) as a negative control for ¹²⁵I-anti-HA IgG endocytosis (Li, Y., J. Biol. Chem. 275: 17187-17194, 2000).

FIG. 6 illustrates that LRP6 is not a constitutively active endocytosis receptor. (A) Anti-HA IgG binding to cell surface HA-tagged LRP6. Binding of ¹²⁵I-anti-HA IgG (1 nM) to LRP6-transduced HT1080 cells and the control cells was carried out for 90 minutes at 4° C. (B) LRP6 endocytosis. LRP6-transduced HT1080 cells, mLRP4-transfected CHO cells and mLRP4tailess-transfected CHO cells were incubated with 1 nM ¹²⁵I-anti-HA IgG at 4° C. for 90 minutes, and then incubated at 37° C. for the indicated times. The amount of internalized anti-HA IgG was determined. Values are the means of triple determinations with the s.d. indicated by error bars. Interestingly, it was found that the endocytosis rate of LRP6 was extremely slow, and was indistinguishable from that of mLRP4tailess (FIG. 6B), indicating that LRP6 itself is unable to initiate endocytosis. From these data, it was concluded that LRP6 is not a constitutively active endocytosis receptor and mediates a limited level of Mesd degradation.

### Example 7

### LRP6 Mediates Little MESD Uptake and Degradation

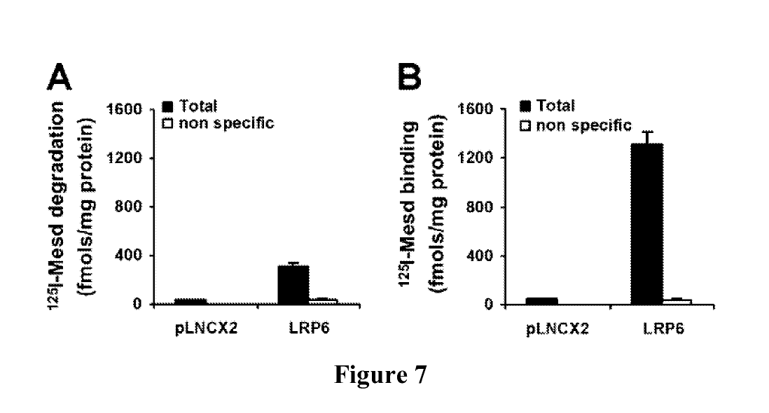

LRP6-mediated Mesd uptake and degradation was investigated in the experiments illustrated in FIG. 7. (A) LRP6-mediated ¹²⁵I-Mesd (5 nM) degradation in LRP6-transduced HT1080 cells and control cells was carried out for 4 hours at 37° C. in the absence or presence of 500 nM Mesd. (B) ¹²⁵I-Mesd (5 nM) binding to LRP6-transduced HT1080 cells and the control cells was carried out for 4 hours at 4° C. in the absence or presence of 500 nM Mesd. Values are the means of triple determinations with the s.d. indicated by error bars. As shown in FIG. 7, HT1080 cells transduced with LRP6 exhibited ¹²⁵I-Mesd degradation at a level of 320 fmoles/mg cell protein after 4 hours of incubation at 37° C., whereas ¹²⁵I-Mesd binding following 4 hours of incubation at 4° C. was detected at a level as high as 1320 fmoles/mg cell protein. These results indicate that Mesd binding to LRP6 at the cell surface does not trigger significant endocytosis, and consequently little Mesd uptake and degradation can be detected.

### Example 8

### MESD Binding to the Cell Surface LRP6 does not Significantly Change the Cytosolic B-Catenin Level

β-catenin is a key molecule in the Wnt/β-catenin signaling pathway. A cytosolic pool of β-catenin interacts with DNA-binding proteins and participates in Wnt signal transduction (Hinck, L., J. Cell Biol. 125, 1327-13401994; Gottardi, C. J., J. Cell Biol. 153: 1049-1060, 2001; Klingelhofer, J., Oncogene 22, 1181-1188, 2003). To determine whether Mesd binding to cell surface LRP6 directly regulates Wnt signaling, the effects of Mesd binding on cytosolic P-catenin levels in HT1080-LRP6 cells were studied. In these experiments, LRP6-transduced HT1080 cells were treated with 0.5 to 5 nM Mesd for 2 hours at 37° C., and cytosolic β-catenin levels were examined by western blotting using an anti-p-catenin antibody. It was found that there was no significant change in the cytosolic β-catenin levels upon Mesd treatment (data not shown). The results indicate that Mesd binding to cell surface LRP6 does not directly modify Wnt signaling.

### Example 9

### RAP Binds to LRP6 and Partially Competes for MESD Binding

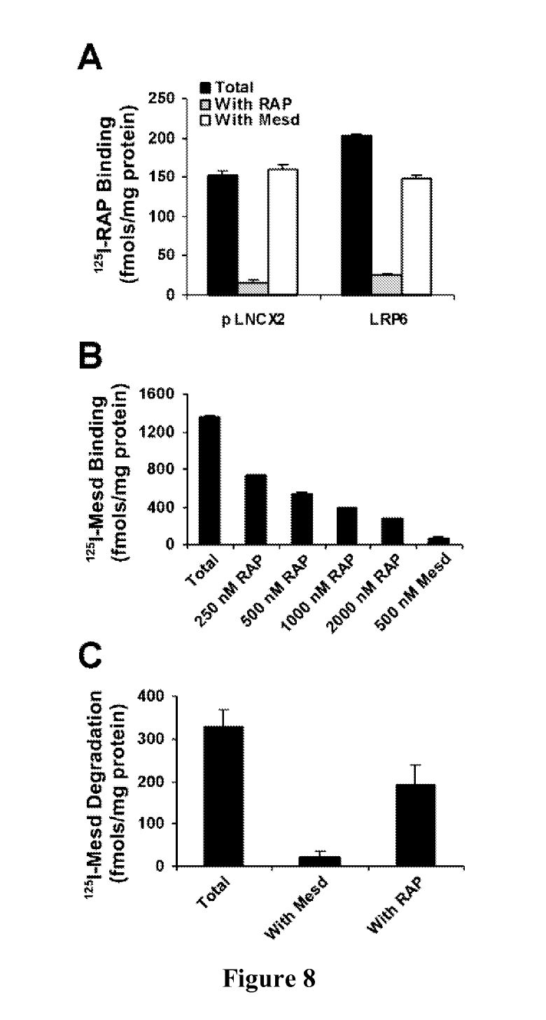

Receptor-associated protein (RAP) binds with high affinity to LRP, megalin, VLDLR and apoER2, and with a lower affinity to the LDLR (Bu, G., Int. Rev. Cytol. 209, 79-116. 2001). To determine whether RAP and Mesd bind to identical, overlapping, or different sites on the receptors, binding and competition analysis of these two chaperones with HT1080 cells stably expressing LRP6 was performed. As shown in FIG. 8, to determine whether RAP also binds LRP6, RAP-binding analysis with HT1080 cells stably expressing LRP6 at 4° C. was performed. Control cells, expressing vector alone, exhibited a moderate level of cell surface ¹²⁵I-RAP binding, probably mediated by cell surface heparan sulfate proteoglycan and endogenous receptors of the LDLR family. The presence of excess unlabeled RAP (500 nM), but not Mesd (500 nm), completely eliminated this binding (FIG. 8A). Compared to the control cells, LRP6 expressing HT1080 cells displayed ˜20% increase of RAP binding, and this increase was abolished by excess unlabeled Mesd (FIG. 8A). These results suggest that RAP binds to cell surface LRP6 with a relatively low affinity.

Next performed was binding of 5 nM ¹²⁵I-Mesd (5 nM) to cell surface LRP6 in the presence of various concentrations of excess unlabeled RAP or 500 nM unlabeled Mesd (FIG. 8B). RAP inhibited ¹²⁵I-Mesd binding in a dose-dependent manner with ˜60% inhibition achieved with 500 nM RAP, whereas the same concentration of unlabeled Mesd inhibited >90% of ¹²⁵I-Mesd binding (FIG. 8B). When ¹²⁵I-Mesd (5 nM) uptake and degradation were performed, 500 nM unlabeled Mesd completely, whereas 500 nM unlabeled RAP only partially, inhibited ¹²⁵I-Mesd degradation (FIG. 8C). Together, these results suggest that Mesd and RAP probably bind to different, but perhaps adjacent sites on LRP6. The lower affinity of RAP to cell surface LRP6 may also contribute to its lower efficiency in inhibition of Mesd binding.

In FIG. 8A, binding of ¹²⁵I-RAP (5 nM) to LRP6-transduced HT1080 cells and the control cells was carried out for 4 hours at 4° C. in the absence (total) or presence of 500 nM RAP, or 500 nM Mesd. In FIG. 8B, binding of ¹²⁵I-Mesd (5 nM) to LRP6-transduced HT1080 cells was carried out for 2 hours at 4° C. in the absence (total) or presence of various concentrations of RAP or 500 nM Mesd. (C) LRP6-mediated ¹²⁵I-Mesd (5 nM) degradation was carried out for 4 hours at 37° C. in the absence or presence of 500 nM Mesd or 500 nM RAP. Values are the means of triple determinations with the s.d. indicated by error bars.

These experiments illustrate that RAP binds to LRP6 and partially competes for Mesd binding.

### Example 10

### MESD Antagonizes Ligand Binding to LRP6 at the Cell Surface

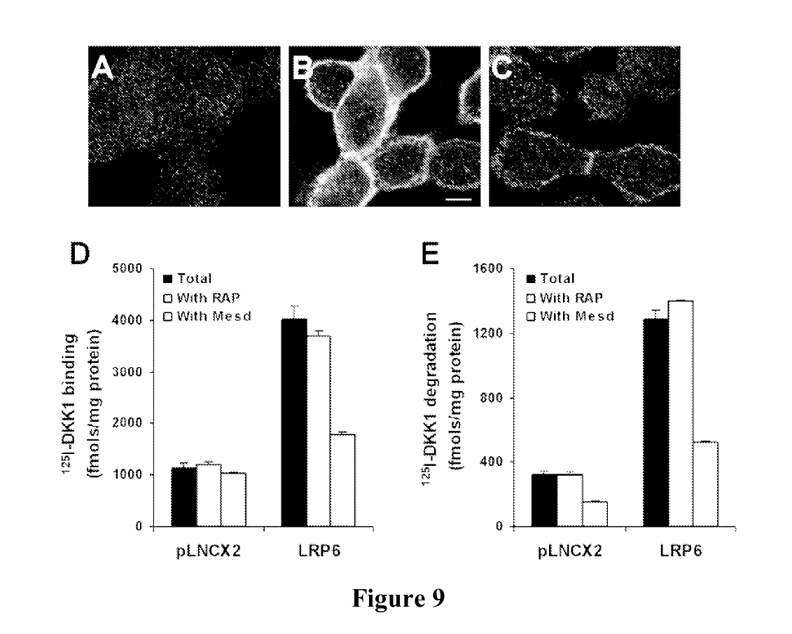

RAP is a receptor antagonist for members of the LDLR family, and is able to inhibit the binding of most known ligands of the LDLR family members. DKK1 is an LRP6-specific ligand and antagonist. To determine whether Mesd is also able to block LRP6 ligand binding, cell surface DKK1 binding by immunostaining was examined. As illustrated in FIG. 9, Myc-tagged DKK1 binds to LRP6 cells (FIG. 9B) but not to the control cells (FIG. 9A). The presence of Mesd completely blocked the binding of Myc-DKK1 to LRP6 at the cell surface (FIG. 9C). In these experiments, Serum-free conditioned medium was harvested from HEK293 cells transiently transfected with cDNA for human Myc-DKK1 and allowed to bind to LRP6-transduced HT1080 cells (B,C) and control cells (A) in the absence (A,B) or presence (C) of 1 μM Mesd. Cell-surface-bound Myc-tagged DKK proteins were fixed and detected by immunofluorescence staining with anti-Myc antibody. (D) DKK1 binding to cell surface LRP6 is inhibited by Mesd. Binding of 125I-DKK1 (5 nM) to LRP6-transduced HT1080 cells or the control cells was carried out for 3 hours at 4° C. in the absence (total) or presence of 500 nM RAP or 500 nM Mesd. (E) LRP6-mediated DKK1 degradation is inhibited by Mesd. LRP6-mediated 125I-Mesd (5 nM) degradation was carried out for 4 hours at 37° C. in the absence or presence of 500 nM RAP or 500 nM Mesd. Values are the means of triple determinations with the s.d. indicated by error bars. Bar, 10 μm. As expected, Myc-tagged DKK1 binds to LRP6 cells (FIG. 9B) but not to the control cells (FIG. 9A). Importantly, the presence of Mesd completely blocked the binding of Myc-DKK1 to LRP6 at the cell surface (FIG. 9C).

To confirm the above results, the binding and degradation of ¹²⁵I-DKK1 was examined. LRP6-expressing HT1080 cells exhibited significantly higher levels of ¹²⁵I-DKK1 binding and degradation than the control cells. The increased DKK1 binding and degradation were abolished by excess unlabeled Mesd, but not by excess unlabeled RAP (FIG. 9D,E). Together, these results indicate that Mesd can specifically block DKK1 binding to LRP6 at the cell surface.

To confirm the above results, the binding and degradation of ¹²⁵I-DKK1 was examined. LRP6-expressing HT1080 cells exhibited significantly higher levels of ¹²⁵I-DKK1 binding and degradation than the control cells. The increased DKK1 binding and degradation were abolished by excess unlabeled Mesd, but not by excess unlabeled RAP (FIG. 9D,E). Together, these results indicate that Mesd can specifically block DKK1 binding to LRP6 at the cell surface.

### Example 11

### Binding of MESD Polypeptide or an MESD Oligopeptide to LRP5 and LRP6

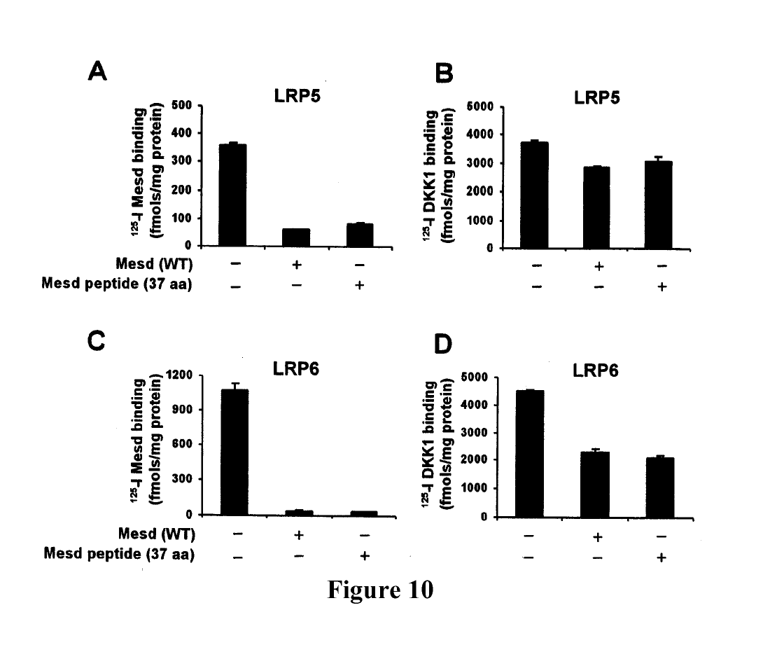

In order to investigate the binding of Mesd polypeptide and an Mesd oligopeptide to either LRP5 or LRP6, binding assays were performed using ¹²⁵I-Mesd or ¹²⁵I-DKK1 (FIG. 10). FIG. 10A shows that both wild type Mesd and Mesd oligopeptide

KGGGSKEKNKTKPEKAKKKEGDPKPRASKEDNRAGSR (SEQ ID NO: 20) can reduce binding of ¹²⁵I-Mesd to LRP5 up to about 10-fold, while FIG. 10C shows that both Mesd and the oligopeptide show even greater reduction of binding of ¹²⁵I-Mesd to LRP6. In addition, in binding assays using ¹²⁵I-DKK1, a small but significant reduction in binding of ¹²⁵I-DKK1 to LRP5 was observed when the LRP5 was contacted with either Mesd or the oligopeptide (FIG. 10B), while binding of ¹²⁵I-DKK1 greater than 2-fold was observed when LRP6 was the target (FIG. 10D).

These data demonstrate that both Mesd polypeptide, and the oligopeptide of sequence SEQ ID NO: 20 can both bind LRP5 and inhibit binding of DKK1 to either LRP5 or LRP6.

### Example 12

### An MESD Oligopeptide Rescues Alkaline Phosphatase Activity in ST-2 Cells Infected with DKK1

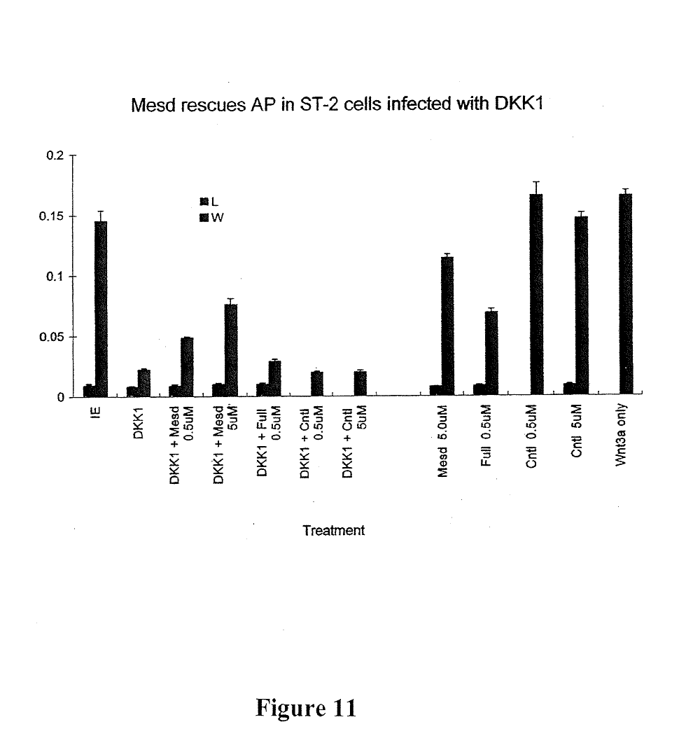

Osteoblasts are cells which build bone. One marker for osteoblast activity is the cell surface enzyme alkaline phosphatase (AP). ST-2 cells are an osteoblast-derived cell line which provides an in vitro model system for studying osteoblast activity. Like osteoblasts in vivo, ST-2 cells exhibit AP activity; AP activity is considered an indicator of osteoblast-like activity in these cells. In order to investigate if Mesd can relieve inhibition of osteoblast activity by DKK1 ST-2 cells, ST-2 cells infected with retrovirus encoding the cDNA of DKK1 were treated with varying levels of Mesd or Mesd oligopeptides, and alkaline phosphatase activity was measured. As shown in FIG. 11, non-transfected ST-2 cells exhibited approximately 0.14 units of AP activity, while AP expression in ST-2 cells infected with DKK1 retrovirus was reduced to about 0.02 units. Treatment of the transfected cells with increasing amounts of a Mesd oligopeptide having sequence

KGGGSKEKNKTKPEKAKKKEGDPKPRASKEDNRAGSR (SEQ ID NO: 20) led to increasing levels of AP activity in the cells. It is concluded, therefore, that contacting osteoblasts with a Mesd oligopeptide can rescue AP activity which is inhibited by DKK1 and hence promote osteoblast function.

### Example 13

### Inhibition of WNT Signalling in HEK293 Cells

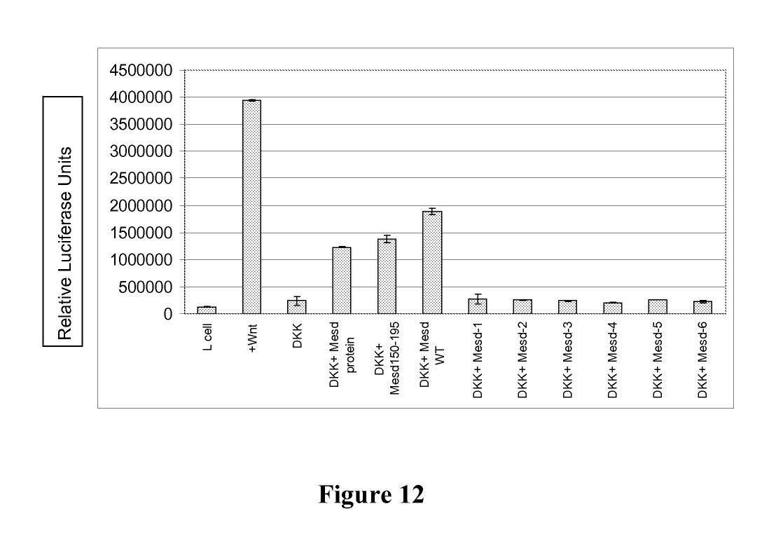

In this example, Wnt signalling was measured in HEK293 cells comprising TCF/LEF-Luc assays in cells were prepared as described above, and subjected to the treatments as shown in Table 2, with the results (i.e., luciferase activity) presented in FIG. 12. These experiments confirm that Dkk1 inhibits Wnt signaling, and show that Mesd protein, Mesd WT peptide

KGGGSKEKNKTKPEKAKKKEGDRKPRASKEDNRAGSR (SEQ ID NO: 21) and Mesd peptide
QMYPGKGGGSKEKNKTKPEKAKKKEGDPKPRASKEDNRAGSRREDL (SEQ ID NO: 22) are each capable of at least partially restoring Wnt signaling inhibited by Dkk1. Peptides Mesd-1 KGGGSKEKNKTKPEKAKKK (SEQ ID NO: 26),
Mesd-2 EGDRKPRASKEDNRAGSR (SEQ ID NO: 24), Mesd-3 TKPEKAKKKEGDRKPRAS (SEQ ID NO: 27), Mesd-4 KGGGSKEKNK (SEQ ID NO: 9), Mesd-5 KEDNRAGSR (SEQ ID NO: 28) and Mesd-6 KEKNKTKPEK (SEQ ID NO: 29) provided controls.

TABLE 2

| | |
|---|---|
| Column | Treatment |

| | |
|---|---|
| 1 | L cell control medium |
| 2 | Wnt3A conditioned medium |
| 3 | Wnt3A conditioned medium plus Dkk1 protein |
| | at 10 nM |
| 4 | Wnt3A conditioned medium plus Dkk1 protein |
| | at 10 nM plus Mesd protein |
| 5 | Wnt3A conditioned medium plus Dkk1 protein |
| | at 10 nM plus Mesd (150-195) peptide, |

(SEQ ID NO: 22)

| | |
|---|---|
| | QMYPGKGGGSKEKNKTKPEKAKKKEGDPKPRASKEDNRAGSR |
| | REDL |
| 6 | Wnt3A conditioned medium plus Dkk1 protein |
| | at 10 nM plus Mesd WT peptide |

(SEQ ID NO: 21)

| | |
|---|---|
| | KGGGSKEKNKTKPEKAKKKEGDRKPRASKEDNRAGSR |
| 7 | Wnt3A conditioned medium plus Dkk1 protein |
| | at 10 nM plus |

(SEQ ID NO: 26)

| | |
|---|---|
| | KGGGSKEKNKTKPEKAKKK (Mesd-1) |
| 8 | Wnt3A conditioned medium plus Dkk1 protein |
| | at 10 nM plus |

(SEQ ID NO: 24)

| | |
|---|---|
| | EGDRKPRASKEDNRAGSR (Mesd-2) |
| 9 | Wnt3A conditioned medium plus Dkk1 protein |
| | at 10 nM plus |

(SEQ ID NO: 27)

| | |
|---|---|
| | TKPEKAKKKEGDRKPRAS (Mesd-3) |
| 10 | Wnt3A conditioned medium plus Dkk1 protein |
| | at 10 nM plus |

(SEQ ID NO: 9)

| | |
|---|---|
| | KGGGSKEKNK (Mesd-4) |
| 11 | Wnt3A conditioned medium plus Dkk1 protein |
| | at 10 nM plus |

(SEQ ID NO: 28)

| | |
|---|---|
| | KEDNRAGSR (Mesd-5) |
| 12 | Wnt3A conditioned medium plus Dkk1 protein |
| | at 10 nM plus |

(SEQ ID NO: 29)

KEKNKTKPEK (Mesd-6)

What is claimed is:

1. A substantially pure oligopeptide consisting essentially of from about 10 contiguous amino acids up to about 70 contiguous amino acids, said oligopeptide comprising an amino acid sequence selected from the group consisting of SEQ ID NO: 9, SEQ ID NO: 10, SEQ ID NO: 11, and SEQ ID NO: 12, and wherein the oligopeptide antagonizes binding of DKK1 to LRP5 when in contact with LRP5.

2. A substantially pure oligopeptide in accordance with claim 1, wherein the oligopeptide consists essentially of from about 20 contiguous amino acids up to about 70 contiguous amino acids.

3. A substantially pure oligopeptide in accordance with claim 2, wherein the oligopeptide consists essentially of from about 30 contiguous amino acids up to about 70 contiguous amino acids.

4. A substantially pure oligopeptide in accordance with claim 1, comprising an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and an amino acid sequence sharing at least 70% sequence identity with at least one sequence set forth as SEQ ID NO: 1 through SEQ ID NO: 29, wherein the oligopeptide antagonizes binding of DKK1 to LRP5 when the oligopeptide is contacted with LRP5.

5. A substantially pure oligopeptide of claim 1, comprising a sequence selected from the group consisting of SEQ ID NO: 20, SEQ ID NO: 21, and SEQ ID NO: 22.

6. A vector comprising a promoter operably linked to a nucleic acid sequence encoding an oligopeptide comprising from about 10 contiguous amino acids up to about 70 contiguous amino acids, wherein the oligopeptide comprises an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20; SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and a sequence sharing at least 70% sequence identity with at least one sequence set forth as SEQ ID NO: 1 through SEQ ID NO: 29, wherein the oligopeptide antagonizes binding of DKK1 to LRP5 when in contact with LRP5.

7. A vector in accordance with claim 6, wherein the oligopeptide comprises a sequence selected from the group consisting of SEQ ID NO: 20, SEQ ID NO: 21, and SEQ ID NO: 22.

8. A cell comprising the vector of claim 7.

9. A method of treating a bone disease, disorder, or injury or promoting bone growth, the method comprising administering to the subject in need thereof a therapeutically effective amount of (i) a Mesd polypeptide or a polypeptide sharing at least 70% sequence identity with a Mesd polypeptide, wherein the polypeptide antagonizes binding of DKK1 to LRP5 when in contact with LRP5; or (ii) an oligopeptide comprising between about 10 contiguous amino acids and about 70 contiguous amino acids, said oligopeptide comprising an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and a sequence sharing at least 70% sequence identity with at least one sequence set forth as SEQ ID NO: 1 through SEQ ID NO: 29, wherein the oligopeptide antagonizes binding of DKK1 to LRP5 when in contact with LRP5.

10. A method in accordance with claim 9, wherein the oligopeptide comprises an amino acid sequence selected from the group consisting of SEQ ID NO: 20, SEQ ID NO: 21, and SEQ ID NO: 22.

11. A method in accordance with claim 9, wherein the Mesd polypeptide has an amino acid sequence selected from the group consisting of SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, and a polypeptide sharing at least 70% sequence identity with a Mesd polypeptide selected from the group consisting of SEQ ID NO: 30 through SEQ ID NO: 38, wherein the polypeptide antagonizes binding of DKK1 to LRP5 when in contact with LRP5.

12. A method in accordance with claim 9, wherein the bone disease, disorder, or injury is at least one selected from the group consisting of bone spurs, bone tumors, bone metastasis, craniosynostosis, enchondroma, fibrous dysplasia, McCune-Albright syndrome, giant cell tumor of bone, Klippel-Feil syndrome, scoliosis, osteitis condensans ilii, osteochondritis dissecans, osteogenesis imperfecta, otospondylomegaepiphyseal dysplasia, Weissenbacher-Zweymüller syndrome, Pallister-Hall syndrome, Greig cephalopolysyndactyly syndrome, McKusick-Kaufman syndrome, Bardet-Biedl syndrome, Oro-facial digital syndromes achondrogenesis, atelosteogenesis, Paget's disease, diastrophic dysplasia, recessive multiple epiphyseal dysplasia, spondyloperipheral dysplasia, ankylosing spondylitis, osteochondroma, osteomyelitis, osteopetroses, renal osteodystrophy, septic arthritis, unicameral bone cyst, osteomalacia, osteoporosis, osteoarthritis, total joint replacement, partial joint replacement, alveolar process-related tooth mobility, alveolar process-related tooth loss, periodontitis, and bone fracture.

13. A method in accordance with claim 9, wherein the bone disease is osteoporosis.

14. A method of treating a bone disease or injury or promoting bone growth, the method comprising administering to a subject in need thereof a therapeutically effective amount of a nucleic acid vector comprising a promoter operably linked to a sequence encoding (i) a Mesd polypeptide or a polypeptide sharing at least 70% sequence identity with a Mesd polypeptide, wherein the polypeptide antagonizes binding of DKK1 to LRP5 when in contact with LRP5; or (ii) an oligopeptide comprising between about 10 contiguous amino acids and about 70 contiguous amino acids, said oligopeptide comprising an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and a sequence sharing at least 70% sequence identity with at least one sequence set forth as SEQ ID NO: 1 through SEQ ID NO: 29, wherein expressed oligopeptide antagonizes binding of DKK1 to LRP5 when in contact with LRP5.

15. A method in accordance with claim 14, wherein the oligopeptide comprises an amino acid sequence selected from the group consisting of SEQ ID NO: 20, SEQ ID NO: 21, and SEQ ID NO: 22.

16. A method in accordance with claim 14, wherein the Mesd polypeptide comprises an amino acid sequence selected from the group consisting of SEQ ID NO: 30, SEQ ID NO: 31, SEQ ID NO: 32, SEQ ID NO: 33, SEQ ID NO: 34, SEQ ID NO: 35, SEQ ID NO :36, SEQ ID NO: 37, SEQ ID NO: 38, and a polypeptide sharing at least 70% sequence identity with a Mesd polypeptide selected from the group consisting of SEQ ID NO: 30 through SEQ ID NO: 38, and wherein expressed polypeptide antagonizes binding of DKK1 to LRP5 when in contact with LRP5.

17. A method in accordance with claim 14, wherein the nucleic acid vector is comprised by a cell.

18. A method in accordance with claim 14, wherein the bone disease is osteoporosis.

19. Use of a polypeptide or an oligonucleotide for the manufacture of a medicament for treatment of a degenerative bone disease, disorder, or injury, wherein the polypeptide or oligonucleotide comprises an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20; SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, and a sequence sharing at least 70% sequence identity with at least one sequence set forth as SEQ ID NO: 1 through SEQ ID NO: 38, wherein the polypeptide or oligonucleotide antagonizes binding of DKK1 to LRP5 when in contact with LRP5.

20. Use of an oligopeptide in accordance with claim 19, wherein the degenerative bone disease is osteoporosis.
